(19) United States
(12) Patent Application Publication
(10) Pub. No.: US 2025/0215389 A1
(43) Pub. Date: Jul. 3, 2025

(54) CULTURE MEDIA BASED ON PROTEIN HYDROLYSATE AND A PROCESS FOR PREPARING THEREOF

(71) Applicant: BTL Healthcare Technologies a.s., Prague (CZ)

(72) Inventors: Jiri Janousek, Jilove u Prahy (CZ); Sara Sediva, Praha 7 (CZ); Marketa Veresova, Praha 8 (CZ); Katerina Hermanova, Praha 4 (CZ); Anna Kohoutova, Praha 6 (CZ)

(21) Appl. No.: 19/029,060

(22) Filed: Jan. 17, 2025

Related U.S. Application Data
(63) Continuation of application No. PCT/IB2024/053805, filed on Apr. 18, 2024.
(60) Provisional application No. 63/497,051, filed on Apr. 19, 2023.

(51) Int. Cl. C12N 5/00

(52) U.S. Cl. CPC C12N 5/0018; C12N 2500/12; C12N 2500/24; C12N 2500/32; C12N 2500/34; C12N 2500/46

(57) ABSTRACT

The present invention provides culture media and method for the cultivation of non-human metazoan cells for preparing a food product.

### CROSS-REFERENCE TO RELATED APPLICATIONS

This application is a continuation of PCT Patent Application No. PCT/IB2024/053805 filed Apr. 18, 2024, which claims priority to U.S. Provisional Patent Application No. 63/497,051 filed Apr. 19, 2023, each of which are incorporated by reference herein in their entireties.

### FIELD OF THE INVENTION

The invention relates to a culture media based on a protein hydrolysate suitable for cell cultivation and a process for preparation thereof. The culture media according to the invention may be used, for example, for cell cultivation for preparing a food product comprising a cell biomass for animal or human nutrition.

### BACKGROUND OF THE INVENTION

Mammalian cells are composed of a variety of chemical compounds. A major component of cell biomass is protein, which usually makes up 60% to 80% of dry mass of cells. Proteins are long polymers of amino acids. There are 20 proteinogenic amino acids, 9 of which are dietary essential in mammals, meaning that they cannot be synthesized by the organism and must be sourced from food, or, in the case of cultivated cells, from the culture media. Additionally, some amino acids may be considered “semi-essential” or conditionally essential, because they can be only synthesized from a particular essential amino acid, or their synthesis tends to be very slow under some conditions.

Cell line-specific spontaneous mutations or deliberate modifications may cause cells to become unable to synthesize one or more amino acids, and therefore said amino acids need to be provided in the culture media (auxotrophy). The cells may, through spontaneous mutations or deliberate modification, gain or regain the ability to synthesize a particular amino acid. Even non-essential amino acids, which the cells are able to synthesize, may be important to the cultivation process, as the cells may also exhibit improved growth or metabolic characteristics when provided with some non-essential amino acids. Cells may also be able to utilize certain other nitrogen sources, notably ammonia, to synthesize non-essential amino acids.

Generally, amino acids mainly serve as building blocks for protein synthesis, and thus are consumed in media in proportion to the amino acid composition of the cellular protein. However, some amino acids may be consumed by the cells at a higher rate than would correspond to their abundance in cellular protein, as they may be used in energetic metabolism, nucleic acid synthesis and other such processes. To provide cells in culture with adequate amino acids for protein synthesis, commonly used culture media formulations contain individual amino acids at different ratios of concentration. These amino acids are usually produced by fermentation processes with microorganisms engineered to produce a specific amino acid. Some amino acids can also be synthesized chemically, but this is generally more expensive than microbial production. However, while microbial production works well for the needs of cell cultivation in research and therapeutic protein production applications, it is generally too expensive for cultivated meat production.

Therefore, there is a need for culture media with an alternative and more economically advantageous source of amino acids. This culture media should be suitable for cell cultivation and economically favorable.

### BRIEF SUMMARY OF THE INVENTION

The disadvantages of the solutions according to state of the art are solved by the present invention that provides culture media suitable for cell cultivation and the processes for preparation thereof.

The culture media may be prepared by dissolving media components in water or a suitable aqueous buffer. The complete medium may be prepared outside of the cell cultivation device, formed for example by a bioreactor, and subsequently introduced to the cultivation device; alternatively, constituent solutions of one or more components may be prepared outside of the cultivation device and introduced separately to the cultivation device; alternatively, the individual components or mixes of components may be introduced to and dissolved directly in the cultivation device; alternatively, a combination of the aforementioned methods may be used.

Sterilization of the culture media is crucial to prevent the contamination of the cultivation device, formed for example by a bioreactor, by undesirable microorganisms. The culture medium may be sterilized after being introduced into the cultivation device; alternatively, the complete culture medium, constituent solutions of one or more components, individual components or their mixes may be sterilized before being introduced into the cultivation device; alternatively, a combination of the aforementioned methods may be used.

The culture media according to the present invention may comprise protein hydrolysate as a source of amino acids. The protein hydrolysate may serve as a source of all amino acids in culture media according to the invention for the purpose of cell cultivation, or some amino acids may be supplied to the media separately. The advantageous process of protein hydrolysis into shorter peptide chains and/or single amino acids is also provided by the present invention.

### BRIEF DESCRIPTION OF THE SEVERAL VIEWS OF THE DRAWINGS

FIG. 1 depicts the cultivation system according to the invention.

### DETAILED DESCRIPTION OF THE INVENTION

The disadvantages of the solutions according to state of the art are solved by the present invention that provides culture media suitable for cell cultivation, and the processes for preparation thereof.

The culture media according to the invention may be used, for example, for cell cultivation for the purpose of using the cell biomass for animal or human nutrition. The culture media according to the invention may be used for cultivated meat production.

The cell types used for cultivation processes in the culture media according to the present invention may comprise many types of non-human metazoan cells, for example, stem cells comprising embryonic stem cells (ESCs) and other cell types derived from blastocyst or other early-stage embryos, muscle stem cells such as myosatellite cells, mesenchymal stem cells or cells derived from the bone marrow, fat tissue, subcutaneous tissue or other tissues, or cells where the stemness character is induced or established afterwards such as induced pluripotent stem cells (iPSCs). Other used cell types may be, for example, myoblasts, myocytes, fibroblasts, fibro-adipogenic progenitors, preadipocytes, adipocytes, epithelial cells, cartilage cells and tendon-derived cells such as chondroblasts and chondrocytes, macrophages, keratinocytes, hepatocytes, testicular cells, Sertoli cells, or any other appropriate cells.

The cell lines used for cultivation processes in the culture media according to the invention may include, for example, Chinese hamster ovary (CHO) cells, for example CHO-K1 or CHO-DG44, C2C12, Madin-Darby bovine kidney cells (MDBKs), Madin-Darby canine kidney (MDCK) cells, UMNSAH/DF-1, or any other appropriate cell lines.

The cells used for cultivation processes in the culture media according to the invention may be any appropriate non-human metazoan cells. The cells for cultivation may be non-human vertebrate cells. The cells may be, for example, bovine, porcine, fish (piscine), game (cervine), avian, rodent (cricetine, murine), equine or any other appropriate cells. The cells for cultivation may be selected, without limitation, from at least one of the following animals: cattle (Bos taurus), chicken (Gallus domesticus), domestic pig (Sus domesticus), house cricket (Acheta domesticus), garden snail (Helix pomatia), common carp (Cyprinus carpio), horse (Equus ferus), edible crab (Cancer pagurus), marsh frog (Pelophylax ridibundus), common octopus (Octopus vulgaris), gilt-head bream (Sparus aurata), roe deer (Capreolus capreolus), common sea urchin (Echinus esculentus), harbor seal (Phoca vitulina), European stag beetle (Lucanus cervus), African elephant (Loxodonta africana), house mouse (Mus musculus), green sea turtle (Chelonia mydas), or from any other appropriate animals.

In one aspect of the invention the cultivated cells may be bovine cells. The bovine cells may be selected from the group of stem cells, comprising embryonic stem cells and other cell types derived from blastocyst or other early-stage embryos, muscle stem cells such as myosatellite cells, mesenchymal stem cells or stem cells derived from bone marrow, fat tissue, subcutaneous tissue or other tissues, or cells where the stemness character is induced or established afterward such as induced pluripotent stem cells. Other used bovine cell types may be bovine myoblasts, myocytes, fibroblasts, fibro-adipogenic progenitors, preadipocytes, adipocytes, epithelial cells cartilage and tendon-derived cells such as chondroblasts and chondrocytes, macrophages, keratinocytes, hepatocytes, testicular cells, Sertoli cells, mesenchymal stem cells, myosatellite cells, or a combination thereof.

In one aspect of the invention, the cells used for cultivation processes in the culture media according to the invention may be in at least one form of: a single cells; cell aggregates, which may take the form of cell clumps (loosely connected aggregates), spheroids (compact homogeneous aggregates) and/or organoids (compact heterogeneous aggregates); cells connected to carriers such as microcarriers, macrocarriers or microfragments; or in any other appropriate form of cells. The cells used for cultivation processes in the culture media according to the invention may be immortalized.

### Culture Media Preparation—General Description

The culture medium may be prepared by dissolving the individual media components in water or a suitable aqueous buffer. The components may be sterilized by a suitable sterilization method in order to remove fungi, bacteria, viruses and other possible contaminating agents. Sterilization may occur before or after dissolving of the medium component. Sterilization may be performed by physical or chemical methods. Chemical methods may include treatment with ozone, chlorine dioxide, ethylene oxide, or any other suitable chemical compound. Physical methods may include treatment with wet or dry heat, ionizing radiation, or any other suitable physical influence. Additionally, for aqueous solutions of media components which are solids, aqueous solutions of media components which are liquids, or separate media components which are liquids, filtration may be used as a method of sterilization. Advantageously, physical methods of sterilization (including filtration) may be used, as they minimize the risk of contamination of the final product with the residues of chemical disinfectants. Advantageously, some medium components or their solutions may be mixed together before sterilization, therefore reducing the number of materials which need to be sterilized separately.

For filter sterilization of a culture medium or its components, the material of the filter may be polyethersulfone, cellulose acetate, ceramic, or any other suitable filter material. The size of the largest pores of the filter may be in the range of 0.001 μm to 10 μm, or in the range of 0.01 μm to 2 μm, or in the range of 0.05 μm to 0.5 μm. Filtration may be carried out as dead-end filtration, tangential flow filtration, alternating flow filtration, or any other suitable filtration setup.

The medium may be stored as the final complete culture medium solution, or as a set of at least one solid component or mixes of solid components, or as a set of at least one concentrated solution of one or more components, or as a set of one or more liquids, or a combination of the above.

The culture medium according to the present invention may be prepared in a culture medium tank. The culture medium tank may comprise at least one of: mixing tank, hydrolysis tank, storage tank, loading tank or waste medium tank, or any other appropriate device.

The media components may be mixed in a mixing tank, which may be made from stainless steel, glass, or any other suitable material. The mixing tank may be equipped with a stirring unit comprising, for example, a shaft with one or more impellers. The mixing tank may be equipped with a heating system. The temperature may be in the range of 10 to 40° C., or in the range of 15 to 38° C., or in the range of 18 to 35° C. The mixing tank may be connected to one or more storage tanks. The mixing tank may be connected to one or more cultivation devices, formed for example by a bioreactor. The culture medium components may be mixed directly in the cultivation device.

The volume of the mixing tank may be in the range of 500 mL to 100 m³, or in the range of 1 L to 10 m³, or in the range of 2 L to 5 m³, or in the range of 500 L to 3 m³.

The storage tanks may be made from stainless steel, glass or any other suitable material. The volume of the storage tank may be in the range of 500 mL to 100 m³, or in the range of 1 L to L to 5 m³, or in the range of 2 L to 3 m³, or in the range of 500 L to 1 m³.

The media components may be dosed into the mixing tank through sterilization filter, or may be sterilized prior to the placement to the mixing tank or may be sterilized in the mixing tank.

The mixing tank may be equipped with different types of sensors, such as, for example, thermal sensor, pH probe, conductometer, or any other type of appropriate sensor according to the needs of the process.

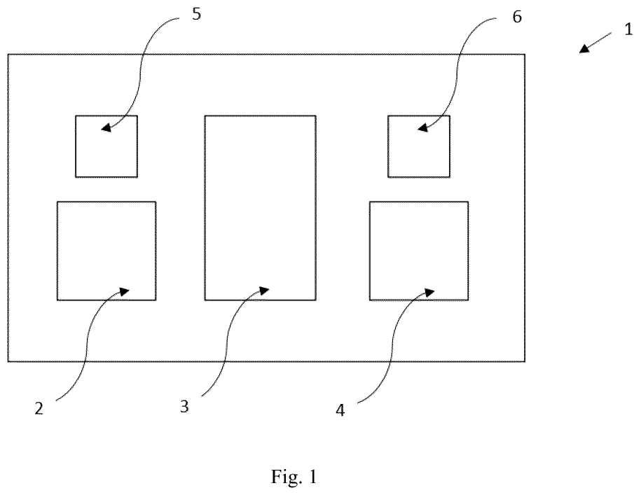

The processes of cell cultivation in culture media according to the present invention may be performed in a cultivation system. In one aspect of the invention, the cultivation system 1 is as depicted in FIG. 1. The cultivation system 1 may comprise a seeding tank 2, a cultivation device 3, a harvesting device 4, a control unit 5, and sensors and analytical instruments 6 as depicted in FIG. 1. Optionally, the cultivation system 1 may further comprise a device for preparing food products (not depicted in FIG. 1).

The control unit may control and/or regulate every process taking place within the cultivation system. The control unit may be operated using at least one printed circuit board (PCB) and/or microprocessor with software capable of controlling the cultivation device, regardless of the extensions and scale of the system. The control unit may be connected to at least one central data storage. The cultivation system may comprise one or more subcontrol units.

The culture media according to present invention may comprise protein hydrolysate as source of amino acids.

The process of medium preparation may further have the characteristics of a batch process, a continuous process or a combination thereof as described below.

### Medium Preparation—Batch Process

In the batch process of medium preparation, the complete culture medium may be prepared, introduced into the cultivation device and subsequently inoculated with cells.

The complete culture medium may be mixed in a mixing tank, or the culture media components may be mixed directly in the cultivation device, as described above.

The complete medium may be sterilized in the mixing tank, in the cultivation device, or during transfer from the mixing tank to the cultivation device, if they are separate. Alternatively, the individual culture media components or mixture of these components may be sterilized before being added to the mixing tank or during transfer to the mixing tank, yielding a sterile complete culture medium.

### Medium Preparation—Continuous Process

In the continuous process of the culture medium preparation, the preparation of the medium may take place simultaneously with the cultivation of cells.

Individual culture medium components or mixture of components may be introduced into the cultivation device during cell cultivation. The components may be sterilized before or while being transferred into the cultivation device. The components may be introduced to the cultivation device in any form, for example they may be introduced as aqueous solutions, allowing convenient filter sterilization and fast mixing of all culture media components in the cultivation device.

The rate of addition of culture media components may be fixed, or the rate of addition may be adjusted to ensure optimal conditions for cell cultivation in the cultivation device. These optimal conditions may include, but are not limited to, the ranges of pH, osmolality, shear protectant concentration, sugar concentration and amino acid concentration as specified herein in the section entitled culture media composition.

Conditions in the cultivation device may be determined by: a pH probe, a conductometer, an osmometer, a glucose prober, a refractometer, an UV-Vis spectrometer, a Raman spectrometer, or any other appropriate measurement method. In appropriate cases, concentration of specific compounds in the cultivation device may also be approximated by a mass balance equation.

To achieve the desired conditions in the cultivation device, the flow rates of medium components may be adjusted using a suitable control mechanism, for example a PID control loop.

In one aspect of the invention, the continuous medium mixing process may comprise the following steps:
introduction of a concentrated basal medium into the cultivation device at a defined flow rate, for example if the basal medium is X times more concentrated than the desired concentration in the final medium, the basal medium is introduced at a flow rate 1/X times the total output flow rate from the cultivation device
introduction of a concentrated sugar solution into the cultivation device wherein the flow rate is controlled so that the concentration of the sugar in the cultivation medium is kept at a desirable setpoint, for example 1 g/L
introduction of a solution of hydrolysate and amino acids into the cultivation device wherein the flow rate is controlled so that the concentration of amino acids in the medium is kept at a desirable setpoint
introduction of a concentrated solution of NaCl into the cultivation device wherein the flow rate is controlled so that the total osmolality of the medium is kept at a desirable setpoint, for example 310 mOsm/kg
introduction of demineralized water into the cultivation device wherein the flow rate is adjusted to be equal to the difference of the total output flow rate from the cultivation device and the sum of all other input flow rates, thereby keeping a constant volume of liquid in the bioreactor.

### Hydrolysate Preparation—General Description

The protein source for hydrolysis may be selected from an industrially scalable protein source. Industrially scalable protein sources include phototrophic organisms, such as land plants, green algae, red algae, brown algae, or other phototrophic eukaryotes, phototrophic prokaryotes such as cyanobacteria, or cultivated heterotrophic prokaryotes or eukaryotes, such as bacteria or yeast. The organism used as a protein source may be able to synthesize all amino acids from inorganic nitrogen sources, such as ammonia ions, nitrate ions or molecular nitrogen. The hydrolysis may be performed, for example, in a hydrolysis tank, or in any other suitable device.

The hydrolysis may be performed on a protein isolate or concentrate from the source organism, or on the whole biomass of the source organism. The source organism may be mechanically or chemically pretreated to improve the speed and efficiency of the hydrolysis process. Saccharides, fats or other compounds may be removed from the biomass of the source organism to facilitate easier processing. Examples of suitable industrially scalable protein sources may include soy, pea, rice, wheat, corn, fava beans, alfalfa, hemp, chickpea, potato, pumpkin, rapeseed, red lentil, Spirulina, Chlorella, sunflower, water lentil, mung bean, flax or baker's yeast. The present invention is not limited to the listed exemplary protein sources.

The protein hydrolysate or multiple hydrolysates from the same or different source organisms may serve as a source of all important amino acids in culture media for the purpose of cell cultivation, or some amino acids may be supplied separately, for example methionine, which is found in very low concentrations in most scalable protein sources. Other different individual amino acids may be supplied separately from a different source than a protein hydrolysate. Typically, methionine and some non-essential amino acids, such as asparagine or glutamic acid, are commercially available wholesale (in food grade) at prices compatible with use in industrial-scale cell cultivation. However, the majority of essential amino acid content of the media according to the invention may be sourced from hydrolysates. The approach may be more economically feasible at large scale than using individual free amino acids, as is commonly done in the biopharmaceutical industry or basic research.

The process of hydrolysis entails breaking the original protein molecule into shorter peptide chains and/or single amino acids. As used herein, the term “protein hydrolysate” is understood to be a mix of amino acids, peptides and other molecules prepared from a suitable protein source by any suitable method, including acidic, basic, or enzymatic hydrolysis, autolysis or lysis by fermentation with a suitable microorganism which is able to break down the protein. The “protein hydrolysate” according to the present disclosure may be, for example, plant protein enzymatic hydrolysates, various types of yeast extracts or lysates (such as whole yeast autolysate), or algae acidic hydrolysate.

Methods of protein hydrolysis may include acidic hydrolysis, basic hydrolysis, enzymatic hydrolysis, or autolysis. Acidic hydrolysis subjects the protein source to a very low pH, usually at an elevated temperature. The duration of reaction may be hours or days. Acidic hydrolysis unfortunately leads to significant degradation of several amino acids, most notably tryptophan, which would then have to be sourced separately at significant costs. Significant degradation of some amino acids also occurs during basic hydrolysis, which subjects the protein source to a very high pH, usually at an elevated temperature. Additionally, the acid or base used for the hydrolysis would have to be removed from the hydrolysate before it could be used to cultivate cells, presenting further complications. For example, when acidic hydrolysis is performed using hydrochloric acid, the acid may be removed by neutralization or evaporation. However, both processes are economically unfavorable because: i) neutralization process results in unfavorably high concentration of salts, which also need to be removed, and ii) evaporation is energy-intensive and the resulting HCl vapors pose a health and environmental hazard that would need to be solved. The process of autolysis relies on the activity of the endogenous enzymes of the source organism to break down the protein source, and this process is usually not very efficient and does not generally result in sufficient hydrolysis of the source protein. Additionally, proteins can be broken down by fermentation with organisms such as Bacillus licheniformis or Aspergillus oryzae, which produce a large amount of proteolytic enzymes. However, with this approach, some of the amino acids from the source protein may be consumed by the organism that was used to break down the protein during the process of fermentation. Also, metabolic waste products and other compounds from the fermenting organism may contaminate the resulting lysate and adversely affect its properties in respect to mammalian cell cultivation.

The hydrolysate according to the invention may be obtained by enzymatic hydrolysis of a suitable protein source. The industrially scalable protein source is advantageous. In one aspect of the invention soy protein isolate may be used as the protein source for enzymatic hydrolysis. Advantageously, soy protein isolate has a favorable ratio of most amino acids for the purpose of mammalian cell cultivation, with the exception of methionine which is present at a relatively low concentration. However, methionine may be added to the media separately as mentioned above.

The protein substrate for hydrolysis in solvent may be subjected to an initial thermal pretreatment to improve solubility and susceptibility to hydrolysis. The temperature during the thermal pretreatment may be in the range of 75 to 95° C., or in the range of 80 to 92.5° C., or in the range of 85 to 90° C. for a time range of 5 to 120 minutes, or 15 to 60 minutes, or for a time range of 30 to 45 minutes.

The method of enzymatic hydrolysis may use a so-called protease, an enzyme that catalyzes the breakdown of peptide bonds in order to achieve protein hydrolysis at much milder conditions than acidic or basic hydrolysis, therefore preserving all of the amino acids of the original protein.

In one aspect of the invention, the enzyme used for hydrolysis may be immobilized on a solid support. This approach sterically prevents the molecules of the enzyme from breaking each other down and allows the enzyme to be separated from the reaction mixture after the reaction and used again. The solid support may be present in the form of solid carriers suspended in the reaction mixture, or a solid structure with a large surface area, such as a sponge or fibrous structure, through which the reaction mixture is perfused. The enzyme may also be added in soluble (free) form. After hydrolysis is complete, the resulting hydrolysate is separated from the solid support with immobilized enzyme by simply draining the reaction vessel (in the case of large solid structure) or removing the enzyme on solid support by filtration or sedimentation (in the case of suspended carriers). The reaction vessel may be formed, for example, by a hydrolysis tank. The filtration step may also remove any solid residues from the source protein, such as cell wall debris. Free enzymes may be removed from the hydrolysate by ultrafiltration or deactivated with elevated temperature when hydrolysis is complete. Ultrafiltration of the hydrolysate may additionally remove any larger peptide chains which were not digested by the enzyme; these peptide chains may be harmful to the cells and therefore their removal may be beneficial. The temperature elevation used to deactivate the enzyme may also sterilize the resulting hydrolysate.

The hydrolysate may be thermally treated at the end of hydrolysis to deactivate enzymes and kill microorganisms. In one aspect of the invention, this treatment may be with lower temperature settings in the range 80 to 120° C., or 85 to 100° C., or in the range 90 to 95° C. with longer time in the range of 15 to 180 minutes, or 20 to 120 minutes, or in the range 25 to 60 minutes. In another aspect of the invention, this treatment may be performed with higher temperature in the range 80 to 160° C., or 100 to 155° C., or in the range 110 to 150° C. with shorter time in the range of 1 to 600 seconds, or 3 to 300 seconds, or in the range of 5 to 60 seconds. Method with lower temperature setting may be performed in reactor setting and both methods may be performed in flash pasteurizer or another suitable continuous flow heating device.

If the enzyme is removed by ultrafiltration, it may retain at least partial catalytic activity and thus may be recycled for another round of hydrolysis. Ultrafiltration or thermal deactivation may also be used to remove active enzyme molecules from hydrolysates prepared by immobilized enzymes, in the event that some of the enzyme detaches from the solid support and dissolves into the reaction mixture.

The solid support may be formed by, for example, silica, epoxide resin, cellulose, chitosan, glass wool, alginate, or by other appropriate materials. The solid support may be in the form of porous or solid beads, sponge, fibers, or another suitable configuration. The solid support may have a large surface area to volume ratio to allow the binding of a large amount of enzyme. For example, beads of porous silica or any other suitable material with a diameter in the range of 1 to 10000 micrometers, or in the range of 10 to 1000 micrometers, or in the range of 20 to 500 micrometers, may be used as a solid support for enzyme immobilization. Immobilization may be achieved, for example, by functionalizing the silica bead surface with amino groups and using a crosslinking agent, such as glutaraldehyde, to bind the enzyme to the solid support. Other functional groups, like aldehyde or epoxy groups, may also be used for enzyme immobilization. The amino groups in this aspect of the invention are covalently bonded to glutaraldehyde, after which excess glutaraldehyde is removed and the enzyme is added. The amino groups on the surface of the enzyme then bind the remaining free aldehyde groups of the glutaraldehyde molecules on the silica bead surface. The immobilization may be performed in water or a suitable aqueous buffer. Thanks to the porous nature and large surface area of the silica beads, a relatively high amount of enzyme may be immobilized relative to the weight of the solid support.

The enzymes according to the invention may be, for example, Alcalase (protease from Bacillus licheniformis), Flavourzyme (protease from Aspergillus oryzae), Protamex, Novo-Pro D, Thermoase PC10FNA, Protease AN Amano 100SD, Protease A Amano 2SD, Protease M Amano SD, Protease P Amano 6SD, ProteAX, Peptidase R, Alkaline Protease, Corolase 7089, Corolase 2TSN, Corolase 8000, Maxipro TNP, Maxipro FPC, Papain, Bromelain, or any other appropriate proteolytic enzyme, or the combination thereof.

Water, or a suitable aqueous buffer, may be used to dissolve the protein source for the hydrolysis. Some proteins may require a buffer to adjust the pH to a level where they have better solubility. The pH may be in the range of 2 to 12, or in the range of 5 to 10, or in the range of 6 to 8.5. A very dilute buffer, or no buffer at all, may be used so that the resulting hydrolysate may be added to the final culture media at high concentrations while minimizing its impact on media osmolarity.

The buffer may include, for example, phosphate buffer, bicarbonate buffer, tris HCl buffer, borate buffer, glycine-NaOH buffer, Good's buffer or any other appropriate buffer, or a combination thereof.

The concentration of the protein in the reaction mixture for hydrolysis may be in the range of 1 to 150 grams per liter, or in the range of 20 to 100 grams per liter, or in the range of 30 to 80 grams per liter of the reaction mixture.

The concentration of the enzyme may be in the range of 0.01 to 10% or in the range of 0.05 to 5%, or in the range of 0.1 to 1% expressed as a ratio of the concentration of enzyme to the concentration of protein in the reaction mixture (further referred to as enzyme/protein ratio). The concentration of the enzyme may be determined by the Bradford assay, BCA assay or other protein determination assays.

In one aspect of the invention, the concentration of potassium phosphate buffer in the range of 1 to 100 mM, or in the range of 10 to 40 mM, or in the range of 15 to 35 mM may be used for pH adjustment to dissolve the soy protein to a concentration in the range of 1 to 150 grams per liter, or in the range of 20 to 100 grams per liter, or in the range of 30 to 80 grams per liter. In another aspect of the invention, the soy protein is dissolved in distilled water to a concentration in the range of 1 to 150 grams per liter, or in the range of 20 to 100 grams per liter, or in the range of 30 to 80 grams per liter.

Other concentrations of the source protein may be used, however very high concentrations of source protein lead to incomplete dissolving of the protein and formation of a highly viscous colloidal solution, presenting problems for the hydrolysis and further processing, while low concentrations of protein may limit the speed of the hydrolysis reaction. To ensure the best dissolution of the proteins in the reaction mixture a heat-treatment may be used. Below boiling temperatures may be used for extended periods of time in order to significantly increase the content of dissolved proteins and to deactivate potential inhibitors of proteases and other antinutritional compounds.

In one aspect of the invention, the source protein may be added at a higher concentration than the maximum soluble concentration. This additional protein may be dissolved after the protein concentration in the reaction mixture is decreased due its hydrolysis by the enzyme. This results in high concentration of available substrate during the entire process, potentially improving hydrolysis efficiency. Multiple cycles of substrate addition into the same reaction mixture may be performed. In one aspect of the invention a base or a suitable buffer may be added to counteract the change and keep the enzyme in its pH optimum or a pH stat may be used

The key parameter by which we can evaluate the conversion of substrate protein into products bioavailable for animal cells is the degree of hydrolysis, defined as the percentage of peptide bonds in the source protein that are hydrolyzed during the reaction. A higher degree of hydrolysis corresponds to a larger percentage of the source protein converted into free amino acids or short peptides, which are usable by mammalian cells as nutrition. Mammalian cells are generally incapable of absorbing and digesting proteins and longer peptides. Peptides longer than four amino acids, or in other words heavier than approximately 500 Daltons, have very poor absorption by mammalian cells. In various aspects of the invention, the amount of the source protein in the range of 20% to 100%, in the range of 30% to 70%, or in the range of 35 to 65%, or in the range of 40% to 60% may be converted into free amino acids, expressed as mass concentration of amino acids to mass concentration of protein. The degree of hydrolysis (DH), meaning the percentage of peptide bonds that undergo hydrolysis out of the total amount of peptide bonds present in the substrate at the start of the reaction, may be in the range of 10% to 60%, in the range 20% to 50%, or in the range of 25% to 40%.

Enzymes used for hydrolysis may fall into two general categories: exoproteases and endoproteases. Exoproteases cleave the protein or peptide chains at the ends, whereas endoproteases can cleave peptide bonds in the middle of the chain. In one aspect of the invention, a combination of endoproteases and exoproteases may be used, since endoproteases may create more free ends of peptide chains, increasing the efficiency of exoproteases, and exoproteases are more efficient in hydrolyzing the protein to single amino acids. In one aspect of the invention, endoproteases and exoproteases may be used sequentially in this order to maximize hydrolysis efficiency.

In one aspect of the invention, additional enzymes may be added to the reaction mixture after the beginning of hydrolysis. This may be done with the same enzyme, mainly in order to counteract the gradual decrease in its enzymatic activity due to degradation of the enzyme molecule. In one aspect of the invention, enzymes with a higher pH optimum may be added at the start of the hydrolysis, when pH is higher, and enzymes with a lower pH optimum may be added later, when the pH is lower, thus maximizing the efficiency of the respective enzymes. The pH tends to decrease naturally during hydrolysis due to the increase in the number of carboxylic groups.

In another aspect of the invention, additional substrate may be added to the reaction mixture after the beginning of hydrolysis. The advantages of this approach may be, for example, easier dispersion and dissolution of additional substrate when the previous amount of substrate is at least partially hydrolyzed.

The addition of enzyme or substrate after the beginning of the hydrolysis process may be performed in a fed-batch (additional reagents are added to the reaction mixture, and subsequently the whole reaction batch is harvested) or in continuous (addition to and harvesting from the reaction mixture are both done continuously) reaction mode.

Regardless of whether immobilized or free enzyme is used, sufficient mixing of the reaction mixture is important to achieve high efficiency. In the case of immobilized enzymes, this applies to both the enzyme immobilization and protein hydrolysis steps. In one aspect of the invention, in the case of immobilized enzymes, mixing methods which minimize mechanical damage to the solid carriers should be used. These may include roller mixing, shaking, or low-shear impellers such as hydrofoil or elephant ear impellers. In the case of enzymes immobilized to a large solid support, sufficient perfusion of the support with the reaction mixture must be assured.

The mixing of the protein source, e.g. protein isolate, with water, or with a suitable aqueous buffer, dissolving the protein source and the process of hydrolysis itself may be performed in an appropriate reaction vessel in a laboratory or industrial scale. The reaction vessel may be formed, for example, by a hydrolysis tank.

The reaction vessel for hydrolysis may comprise, for example, a batch reactor, continuous stirred tank reactor, or plug flow reactor. The volume of the reaction vessel may be in the range of 0.1 L to 100,000 L, or in the range of 0.3 L to 15 000 L, or in the range of 1 L to 5,000 L.

The mixing may be provided by the appropriate stirring unit that may comprise, for example, a paddle impeller. The elephant-ear impeller may be used. The outer diameter of stirrer or impeller may be in the range of 1/10 to 9/10 of the inner reactor diameter, or in the range of 3/10 to 8/10 of the inner reactor diameter, or in the range of 4/10 to 7/10 of the inner reactor diameter, for example ⅔ of the inner reactor diameter. The stirrer or impeller may be located in the center of the reaction vessel or outside of the center of the reaction vessel.

The reaction components may be added to the reaction vessel manually, or based on gravity from the storage tank connected to the reaction vessel, or using a pumping system. The source protein may be in a liquid solution or in a form of powder and may be added to the reaction vessel manually or automatically.

The storage tank may be made, for example, of stainless steel or glass. The volume of the storage tank may be in the range of 100 ml to 5 m³, or in the range of 2 L to 3 m³, or in the range of 500 L to 1 m³.

The reaction vessel, for example a hydrolysis tank, may be equipped with different types of sensors, such as, for example, thermal sensor, pH probe, conductometer, or any other type of appropriate sensor according to the needs of the process of hydrolysis. The pH may be monitored during the whole procedure by a pH electrode. The temperature in the reaction vessel may be regulated, for example, with a reactor thermal jacket, which may be equipped with a heating coil and/or heating/cooling medium.

For precise monitoring of the degree of hydrolysis a sampling system may be used. The degree of hydrolysis may be monitored by titration and/or by absorbance measurement, for example at a wavelength in the range of 190 to 350 nm, or 190 to 230 nm.

After the hydrolysis process, another treatment may be used to reduce the content of phytic acid. Phytic acid is an important compound in plant metabolism, its salt form—phytin is the main storage compound of phosphorus in plants. On the other hand, phytic acid represents one of the antinutrient compounds in legumes, which can significantly influence the downstream processes as well as cell proliferation and viability. An enzymatic treatment or any other method to reduce the content of phytic acid may be used. If enzymatic treatment is used, the selection of the proper phytase enzyme is crucial as well as the proper conditions in regards to pH and temperature. The process may comprise addition of the phytase enzyme in concentration correlation to Enzyme/Substrate ratio, where substrate means the source of protein used. The Enzyme/Substrate ratio may be in the range of 1.1·10⁻¹¹% to 1%, or in the range of 1.1·10⁻¹⁰% to 0.001%, or in the range of 1.1·10⁻⁹% to 0.0001%. The temperature may be in the range of 20° C. to 80° C., in the range of 30° C. to 70° C., or in the range of 40° C. to 60° C. After the enzymatic treatment for time period in the range of 20 minutes to 4 hours, or in the range of 30 minutes to 3 hours, or in the range of 1 to 2 hours, the heat-treatment for enzyme deactivation may be applied. Deactivation may be performed for example for 30 minutes at 90° C., or 15 minutes at 95° C.

For the purpose of filtration, for example for removing impurities, for separation of enzyme immobilized on a carrier from the reaction mixture, or for separation of larger peptides from hydrolysate, appropriate filtration unit equipped with filtration materials may be used. The filtration material may be, for example, filtration fabrics, ceramics, glass, membranes or other suitable materials. The size of pores in filtration material may be for example, but not limited to, 500 μm-10 μm for filtration, 10 μm to 0.1 μm for microfiltration, 0.1 μm to 1 nm for ultrafiltration and 1 nm to 0.1 nm for nanofiltration. The membranes characterized with the range of 60 kDa to 500 Da may be used. As a prior step to filtration, centrifugation may be used to ease the process of filtration.

### Hydrolysis by Free Enzyme

In one aspect of the invention, hydrolysis by free enzymes may be performed by dissolving the protein substrate in the reaction vessel formed, for example, by a hydrolysis tank. This protein substrate may be, for example, whole biomass, protein concentrate, protein flour, raw protein meal, protein extractor protein isolate from soy, pea, rice, wheat, corn, fava beans, alfalfa, hemp, chickpea, potato, pumpkin, rapeseed, red lentil, Spirulina, Chlorella, sunflower, water lentil, mung bean, flax or yeast, or another suitable protein source.

The concentration of protein in the reaction mixture may be in the range of 1 g/L to 150 g/L, or in the range 20 g/L to 100 g/L, or in the range of 30 to 80 g/L. For a given volume of the reaction mixture, the Alcalase may be added in calculated amounts accordingly to the Enzyme/Protein ratio in the range of 0.01 to 10%, or in the range of 0.05 to 3%, or in the range of 0.1 to 0.8% of the concentration of the enzyme/the concentration of the protein. The resulting mixture has a neutral to basic pH, allowing for a high activity of Alcalase. The temperature may be in the range of 50° C. to 70° C., or in the range 55° C. to 65° C., or in the range of 58° C. to 63° C. Over a period of constant mixing, which may be in the range of 30 minutes to 24 hours, or in the range of 1 to 12 hours, or in the range of 2 to 8 hours, the pH of the mixture decreases as the results of the hydrolysis of peptide bonds and increased number of carboxylic groups.

This allows for a high activity of Flavourzyme, which may be added in calculated amounts accordingly to the Enzyme/Protein ratio in the range 0.01 to 10%, or in the range 0.1 to 2%, or in the range of 0.2 to 1% Enzyme/Protein ratio to the reaction mixture. The resulting mixture may then be incubated for an additional time period in the range of 1 hour to 48 hours, or in the range of 5 to 24 hours, or in the range of 8 to 20 hours at temperature in the range of 30 to 80° C., or in the range of 40° C. to 70° C., or in the range of 45 to 60° C., with constant mixing, after which the residual enzyme is thermally deactivated. With this procedure, 20% to 100%, 30% to 70%, or 40% to 60% of the source protein may be converted into free amino acids.

### Hydrolysis by Immobilized Enzyme

The protein hydrolysis process may be carried out in the reaction vessel formed, for example, by a hydrolysis tank. The protein hydrolysis may be carried out with immobilized enzyme in an amount in the range of 0.01 g to 10 g, or in the range of 0.25 to 1.8 g, or in the range of 0.5 to 1.5 g on 10 grams of enzyme carrier. The enzyme carrier may be made from glass, porous silica, alginate, epoxy methacrylate, chitosan, or from any other suitable material, in the form of beads, wool, sponge, fibers, or in any other suitable form. The enzyme carrier may be, for example, formed by glass beads, porous silica beads, alginate beads, epoxy methacrylate beads, glass wool, chitosan, or any other suitable enzyme carrier. Suitable enzyme carriers are described in more detail in the section herein entitled “Hydrolysate preparation—general description”. For example, 1 gram of immobilized enzyme on 10 grams of porous silica beads may be used.

The immobilized enzymes may be prepared by suspending a set weight of NH₂-functionalized porous silica microbeads in the set weight of distilled water. The ratio of set weight of NH₂-functionalized porous silica microbeads versus distilled water may be in the range of 1:1 to 1:10000, or in the range of 1:10 to 1:1000, or in the range of 1:20 to 1:100. Silica beads are further activated with the addition of glutaraldehyde. The amount of glutaraldehyde added to the reaction mixture may be in the range of 0.01 to 70 mmol, or in the range of 0.05 to 40 mmol, or in the range of 0.1 to 10 mmol of glutaraldehyde per 1 g of silica beads. The excess glutaraldehyde is washed away, and the silica beads are resuspended, for example, in half the original volume. Alcalase is then added to a final concentration with constant stirring. This procedure may immobilize 10 to 100%, 60 to 90%, or 70 to 80% of the used enzyme on the silica beads. This may correspond to 10 to 100 grams, 30 to 60, or 40 to 50 grams of enzyme immobilized per 1 kilogram of silica beads.

In one aspect of the invention, silica beads with immobilized Alcalase may be added to a mixture of a soy protein and distilled water. The amount of silica beads with immobilized Alcalase may be, for example, in the range of 10 to 20 g/L, or in the range of 12 to 18 g/L, or in the range of 14 to 16 g/L, or any other appropriate amount. After hydrolysis with Alcalase the beads bound to Alcalase may be removed by centrifugation. Silica beads with immobilized Flavourzyme are added in an amount, for example, in the range of 4 to 40 g/L, or in the range of 5 to 30 g/L, or in the range of 10 to 20 g/L. The appropriate time of hydrolysis may be, for example, in the range of 10 minutes to 24 hours, or in the range of 30 minutes to 12 hours, or in the range of 1 to 6 hours.

The temperature of hydrolysis may be in the range of 10 to 90° C., or in the range of 25 to 80° C., or in the range of 50 to 70° C. In another aspect of the invention, Alcalase beads may not be removed at this step and may instead be removed at the end of the process. In yet another aspect of the invention, Alcalase and Flavourzyme beads may have different sizes, facilitating their separation after removal from the solution. In another aspect of the invention, Flavourzyme beads may be added at the start of hydrolysis or at any other point during the hydrolysis. After further hydrolysis, for a time period which may be in the range of 1 to 24 hours, or in the range of 6 to 20 hours, or in the range of 10 to 18 hours, at a temperature which may be in the range of 20 to 90° C., or in the range of 30 to 80° C., or in the range of 40 to 60° C., with constant mixing, the Flavourzyme beads are removed by centrifugation and the resulting hydrolysate is thermally sterilized, which also deactivates any enzyme which could have detached from the solid support. After filtration to remove solid debris, the hydrolysate can be used to prepare culture media. With this method, the amount of source protein in the range of 20 to 100%, or in the range of 30 to 95%, or in the range of 40 to 90%, may be converted into cell-usable products, meaning free amino acids or peptides of 500 Da or less.

Since Alcalase and Flavourzyme are quite stable in their immobilized form, they may be recycled in the hydrolysate production process according to the invention. In one aspect of the invention, the silica beads with immobilized Alcalase may be used for 2 to 50, 5 to 40, or 10 to 30 cycles of hydrolysis while maintaining around half of their original catalytic activity. In another aspect of the invention, silica beads with immobilized Flavourzyme can be used for 2 to 50, 5 to 40, or 10 to 30 hydrolysis cycles while maintaining sufficient catalytic activity. Generally, even though immobilized enzymes tend to be more stable than free enzymes, their enzymatic activity decreases with use. Therefore, in later cycles, duration of the reaction or enzyme to protein ratio may be changed to maintain a consistent quality of the resulting hydrolysate.

### Culture Media Composition

In one aspect of the invention, the composition of the culture medium may be defined in terms of the total input of medium components into the cultivation process. In this aspect of the invention, summary amounts of components introduced into the cultivation process at any time point over its entire duration are provided. Furthermore, in this aspect of the invention, the provided concentration ranges for the individual medium components describe the total amount of the given component introduced into the cultivation process at any time point during the cultivation process in relation to the volume of spent culture medium which exits the process. The spent culture medium may exit the cultivation process together with the cultivated cells (harvesting), or separately from the cultivated cells (perfusion). The cultivation process may further have the characteristics of a batch process, where all of the components are introduced into the cultivation process at a single time point and the harvest is performed at a single time point, a fed-batch process, where some components may be introduced after the start of the process and the harvest is done at a single time point, a continuous process, where components may be introduced during the whole duration of cultivation and harvesting may be performed during the whole duration of cultivation, or a combination of the described characteristics. For brevity, this aspect of the invention will be referred to herein as “total input”.

In another aspect of the invention, the composition of the culture medium may be described in terms of the concentration of components which are present at a particular time point during the cell cultivation process in the culture medium. In this aspect of the invention, the provided concentration ranges for the individual medium components describe the concentrations present in the culture medium in the cultivation device at any time point during the cultivation process. For brevity, this aspect of the invention will be referred to herein as “momentary composition”.

### Total Input

The total inputs into the culture medium according to the invention may comprise an optimized ratio essential of amino acids, which may be sourced from a protein hydrolysate, in combination with at least one type of compound selected from a group comprising: sugars, vitamins and organic micronutrients, mineral compounds, iron supplementation compounds, organic amines, and shear protectants, or a combination thereof. The media may also contain other compounds, like fatty acids, phospholipids, or nucleic acids, for example. Media according to the invention with an optimized ratio of amino acids and other nutrients may facilitate efficient production of biomass and a low production of waste metabolites, such as ammonia or lactate, by the cells.

An optimized ratio of essential amino acids is such that essential amino acids may be introduced into the cultivation process in any ratio where the percentage of essential amino acids that can be converted into cellular protein is in the range of 5% to 100%, or in the range of 20 to 90%, or in the range of 30 to 80%. The term “highest possible conversion efficiency” determines what percent of the essential amino acids provided to the cells can be converted into cellular protein, assuming no loss of amino acids to catabolism, conversion to other compounds (nucleic acids, for example), or spontaneous degradation.

The highest possible conversion efficiency is determined by the essential amino acid that is the most limiting to the cells. It is calculated such as that for all individual essential amino acids added to the medium in any form at any time point during the cultivation process, the content of that particular essential amino acid in the culture media as a fraction of total essential amino acid content added in any form at any time point to the culture media is divided by the content of that individual amino acid in cellular protein as a fraction of total content of essential amino acids in the lowest obtained ratio, in other words the ratio for the essential amino acid which forms the lowest percentage of the amino acids added to the medium in comparison to the percentage of that particular amino acid in cellular biomass, is then multiplied by 100 to obtain the highest possible conversion efficiency of the provided essential amino acids into cellular protein. All percentages in the calculation of highest possible conversion efficiency are percentages by weight.

The amino acids in the culture media may be present in the form of free amino acids or peptides. Non-essential amino acids are omitted in this calculation, as they can be synthesized by the cells and thus are not limiting in terms of the highest possible conversion efficiency. An example of possible essential amino acid content in cellular protein can be seen in Table 1 below.

The above description may be summarized by the following equation:

$${H}_{EAA} = \frac{\frac{{A}_{EAAM}}{\sum {A}_{EAAM}}}{\frac{{A}_{EAAC}}{\sum {A}_{EAAC}}} * 100 ,$$

where
H_{EAA} is the highest conversion efficiency for a particular amino acid,
A_{EAAM} is the content of that particular essential amino acid in 100 g of protein in culture medium, ΣA_{EAAM} is the total content of all essential amino acids in 100 g of protein in culture medium,
A_{EAAC} is the content of that particular essential amino acid in 100 g of cellular protein and
ΣA_{EAAC} is the total content of all essential amino acids in 100 g of cellular protein.

An example calculation for the essential amino acid tryptophan would proceed as follows: assuming that the total amount of tryptophan added to the culture media over the period of cultivation was 2 grams, and the total amount essential amino acids added to the media over the same time period was 100 grams. Table 1 shows that in 100 grams of cellular protein, out of 44.7 grams of total essential amino acids, 1.6 grams are tryptophan.

### The Calculation:

$${H}_{EAA} = \frac{\frac{2}{100}}{\frac{1.6}{44.7}} * 100 = 55.875 \%$$

shows that the highest conversion efficiency for tryptophan is 55.875%. Now, this process is repeated for each of the nine individual essential amino acid. The lowest of nine numbers obtained is the final highest conversion efficiency.

The amount of essential amino acids that can be converted into cellular protein is determined by how closely the total input of essential amino acids into the cultivation process matches the amino acid composition of cellular protein. Because cells cannot synthesize essential amino acids, the essential amino acid with the lowest relative total input into the cultivation process in comparison to its content in cellular protein will limit maximal cell yield and therefore the maximal percentage of essential amino acids converted to cellular protein (this can be understood as an application of Liebig's law of the minimum).

The conversion efficiency for total essential amino acids may be in the range of 5% to 100%, 20% to 100%, 30% to 100%, or 50% to 100%, calculated by the above mentioned equation.

If the essential amino acid composition of cellular protein according to the example mentioned in Table 1 is used, the resulting total inputs of each essential amino acid given as grams per 100 grams of the total input of all essential amino acids may be in the ranges summarized in the Table 2.

The ranges of concentrations of amino acids in grams per 100 grams of total essential amino acids introduced into the cultivation process may be according to Table 2, regardless of whether the essential amino acid composition of cellular protein is according to Table 1 or not.

It should be noted that for the purpose of this equation, it is necessary to consistently consider amino acid content either as free amino acids, or as amino acids that are part of a peptide chain (in which case the molecular weight of each amino acid must be considered lower by the weight of one water molecule, to account for the fact that water is a byproduct of a peptide bond formation). In the equation above and Tables 1-3, everything is counted as amino acids that form a peptide chain. Elsewhere in the present document, when amino acid input or concentration is discussed, these are calculated with the molecular weights of free amino acids, and when protein input or concentration is discussed, it is assumed that the amino acids are part of a peptide chain for any calculations.

TABLE 1
example of possible essential amino
acid content in the cellular protein.

| | content [g/100 g |
|---|---|
| Amino acid | cellular protein] |
| His | 2.7 |
| Ile | 5.1 |
| Leu | 8.9 |
| Lys | 8.2 |
| Met | 2.9 |
| Phe | 4.7 |
| Thr | 4.8 |
| Trp | 1.6 |
| Val | 5.8 |
| Sum | 44.7 |

TABLE 2
ranges of concentrations of amino acids in grams per 100 grams of total
essential amino acids introduced into the cultivation process.

| Amino acid | range 1 | range 2 | range 3 |
|---|---|---|---|
| His | 0.30 to 6.04 | 1.21 to 5.44 | 1.81 to 4.83 |
| Ile | 0.57 to 11.41 | 2.28 to 10.27 | 3.42 to 9.13 |
| Leu | 1.00 to 19.91 | 3.98 to 17.92 | 5.97 to 15.93 |
| Lys | 0.92 to 18.34 | 3.67 to 16.51 | 5.50 to 14.68 |
| Met | 0.32 to 6.49 | 1.30 to 5.84 | 1.95 to 5.19 |
| Phe | 0.53 to 10.51 | 2.10 to 9.46 | 3.15 to 8.41 |
| Thr | 0.54 to 10.74 | 2.15 to 9.66 | 3.22 to 8.59 |
| Trp | 0.18 to 3.58 | 0.72 to 3.22 | 1.07 to 2.86 |
| Val | 0.65 to 12.98 | 2.60 to 11.68 | 3.89 to 10.38 |

However, the composition of cell biomass is somewhat variable, and therefore the values for each essential amino acid in terms of weight percentage of total essential amino acids used in the media may also be in the ranges summarized in the Table 3.

TABLE 3
ranges of weight percentage concentration of total essential
amino acids introduced into the cultivation process.

| Amino acid | range 4 | range 5 | range 6 |
|---|---|---|---|
| His | 0.2 to 7.9 | 0.8 to 7.1 | 1.2 to 6.3 |
| Ile | 0.3 to 14.9 | 1.5 to 13.4 | 2.3 to 11.9 |
| Leu | 0.7 to 25.9 | 2.7 to 23.3 | 4.1 to 20.8 |
| Lys | 0.6 to 23.9 | 2.5 to 21.5 | 3.8 to 19.1 |
| Met | 0.2 to 8.5 | 0.9 to 7.6 | 1.3 to 6.8 |
| Phe | 0.3 to 13.7 | 1.4 to 12.3 | 2.2 to 11.0 |
| Thr | 0.3 to 14.0 | 1.5 to 12.6 | 2.2 to 11.2 |
| Trp | 0.1 to 4.7 | 0.5 to 4.2 | 0.7 to 3.8 |
| Val | 0.4 to 16.9 | 1.8 to 15.2 | 2.7 to 13.5 |

Amino acids may be introduced into the cultivation process in the form of free amino acids, salts of amino acids, esters of amino acids, or any other suitable derivatives, as well as oligopeptides, for example dipeptides, tripeptides or tetrapeptides, or polypeptides. The culture medium according to the invention may comprise soy protein enzymatic hydrolysate, or any other appropriate scalable hydrolysate according to the description of hydrolysates and preparation thereof, as mentioned above. For example, the suitable industrially scalable protein sources for hydrolysate preparation may include soy, pea, rice, wheat, corn, fava beans, alfalfa, hemp, chickpea, potato, pumpkin, rapeseed, red lentil, Spirulina, Chlorella, sunflower, water lentil, mung bean or yeast. The present invention is not limited to the listed exemplary protein sources.

The total input of hydrolysate (expressed as protein dry weight) introduced into the culture medium in the cultivation process may be in the range of 1 g/L to 200 g/L, or in the range of 3 g/L to 100 g/L, or in the range of 10 g/L to 60 g/L, or in the range of 8 g/L to 50 g/L.

The total input of amino acids from hydrolysate, including amino acids in the form of short peptides or suitable bioavailable derivatives, for example phosphoesters, such phosphoserine, or other derivatives, such as methylglycine, is at least 75%, 80%, 85%, 90%, or 95% by weight of the total input of all amino acids into the culture medium.

The culture medium according to the invention may comprise amino acids added separately from the hydrolysate, for example L-methionine, L-cysteine or L-ornithine. The total input of amino acids added separately from hydrolysate may be in the range of 0.02 g/L to 30 g/L, or in the range of 0.05 g/L to 15 g/L, or in the range of 0.1 g/L to 10 g/L.

The total amount of L-cysteine in culture medium may be in the range of 0.1 to 10, or 0.5 to 7, or 1 to 5% by weight with respect to the total amount of hydrolysate protein in the culture medium.

The total amount of L-ornithine in culture medium is in the range of 0 to 5, or 0.0001 to 3, or 0.001 to 0.5% with respect to the total amount of hydrolysate protein in the culture medium.

The total amount of L-methionine in culture medium may be in the range of 0.05 to 6, or 0.1 to 3, or 0.2 to 2% with respect to the total amount of hydrolysate protein in the culture medium.

The total amount of L-tryptophan in culture medium may be in the range of 0.05 to 6, or 0.1 to 3, or 0.2 to 2% with respect to the total amount of hydrolysate protein in the culture medium.

The total amount of L-histidine in culture medium may be in the range of 0.03 to 4, or 0.07 to 2, or 0.15 to 1.5% with respect to the total amount of hydrolysate protein in the culture medium.

The total amount of L-threonine in culture medium may be in the range of 0.1 to 7, or 0.2 to 5, or 0.3 to 3% with respect to the total amount of hydrolysate protein in the culture medium.

The total input of amino acids added to the culture medium separately from the hydrolysate may be in the range of 0.2% to 25%, or in the range of 0.5 to 15%, or in the range of 1 to 10%, expressed as a percentage of the total input of hydrolysate protein into the culture medium.

The culture medium according to the invention may comprise an inorganic source of bioavailable nitrogen, for example ammonia. The total input of inorganic nitrogen source may be in the range 0 g/L to 30 g/L, or in the range 0.5 g/L to 20 g/L, 1 g/L to 10 g/L.

As a sugar may be used at least one compound selected from the group: glucose, fructose, galactose, sucrose, lactose, maltose, or a combination thereof, or any other appropriate saccharide. Total input of sugars may be in an amount in the range of 1 g/L to 350 g/L, or in the range of 2 g/L to 100 g/L, or in the range of 3 g/L to 20 g/L.

The media may contain at least one of or any combination of the following ions as a mineral compound: Ca²⁺, Cl⁻, Cu²⁺, SO₄²⁻, Fe³⁺, NO³⁻, Fe²⁺, Mg²⁺, K⁺, Na⁺, CO₃²⁻, HCO₃⁻, H₂PO₄⁻, HPO₄²⁻, PO₄³⁻, Zn²⁺, SeO₃²⁻. The media may also contain trace amounts of other mineral compounds and elements, such as cobalt, iodine or manganese.

As the media is prepared by dissolving different constituent compounds in water, any appropriate chemical compound may be used as long as it dissociates to the desired ions in aqueous solution. For example, NaCl and KCl both produce a Cl⁻ ion when dissolved. As another example, CuSO₄ and MgCl₂ or MgSO₄ and CuCl₂ may be used to produce Cu²⁺, Mg²⁺, SO₄²⁻ and Cl⁻ ions. Assuming equimolar amounts, the resulting aqueous solution will have the same composition for both combinations of compounds used. The total input of mineral compounds introduced into the cultivation process may be in the range of 0.1 g/L to 50 g/L, or in the range of 1 g/L to 20 g/L, or in the range of 3 g/L to 10 g/L.

The total input of Na⁺ may be in the range of 20 to 120 mmol/L, or in the range of 30 to 100 mmol/L, or in the range of 40 to 80 mmol/L.

The total input of Cl⁻ may be in the range of 25 to 130 mmol/L, or in the range of 35 to 110 mmol/L, or in the range of 45 to 90 mmol/L.

The total input of Mg²⁺ may be in the range of 0.3 to 10 mmol/L, or in the range of 0.5 to 8 mmol/L, or in the range of 1 to 5 mmol/L.

The total input of PO₄³⁻ may be in the range of 0.5 to 12 mmol/L, or in the range of 0.7 to 10 mmol/L, or in the range of 1 to 6 mmol/L.

The total input of SO₄²⁻ may be in the range of 0.1 to 5 mmol/L, or in the range 0.3 to 3 mmol/L, or in the range 0.6 to 2 mmol/L.

The total input of K⁺ may be in the range of 2 to 18 mmol/L, or in the range of 4 to 15 mmol/L, or in the range of 6 to 12 mmol/L.

The media may contain at least one vitamin of: alpha-tocopherol (vitamin E), ascorbic acid (vitamin C), vitamin B12, biotin, choline, pantothenic acid, folic acid, niacinamide, pyridoxine, riboflavin, thiamine, i-inositol, or a combination thereof. Any appropriate bioactive derivatives or precursors of these compounds may be used. For example, cyanocobalamin may be used instead of vitamin B12, as it can be readily converted to bioactive vitamin B12 by the cells. As another example, thiamine hydrochloride (chloride salt form of thiamine) may be used instead of thiamine. The total input of vitamins introduced into the cultivation process, omitting the vitamins present in lysates or extracts, may be in the range of 0.1 mg/L to 1,000 mg/L, or in the range of 5 mg/L to 500 mg/L, or in the range of 20 mg/L to 300 mg/L.

The total input of choline may be in the range of 10 mg/L to 1,000 mg/L, or in the range of 20 mg/L to 500 mg/L, or in the range of 30 mg/L to 200 mg/L.

The total input of niacinamide (or another vitamer of vitamin B3) may be in the range 3 mg/L to 150 mg/L, or in the range 6 mg/L to 100 mg/L, or in the range of 10 mg/L to 80 mg/L.

As an organic amine may be used at least one compound selected from: putrescine, ethanolamine, or a combination thereof, or any other appropriate amine. Organic amines total input into the cultivation process may be in an amount in the range of 0.01 mg/L to 1,000 mg/L, or in the range of 0.1 mg/L to 100 mg/L, or in the range of 0.5 mg/L to 20 mg/L.

Vitamins and organic amines or their respective precursors or derivatives may be supplied in the form of a lysate or extract, for example autolysed yeast extract or any other appropriate lysate or extract. Extract or lysate for supplementation of micronutrients may be added to the culture media in an amount in the range of 0.01 g/L to 20 g/L, or in the range of 0.1 g/L to 10 g/L, or in the range of 0.5 g/L to 5 g/L.

Iron may be supplemented to the culture medium in compounds with oxidation state iron(III) or iron(II). Iron may be present as free ions, or it may be chelated with a suitable chelating agent to improve its solubility and bioavailability. Chelating agents may include citrate, gluconate, ammonium citrate, EDTA, their combinations, or any other suitable chelating agent. Iron may be introduced into the culture medium bound to the chelating agent (for example, in the form ferric citrate), or iron and the chelating agent may be added separately (for example, in the form of ferric chloride and sodium citrate). The relative amount (w/w) of the total input of the chelating agent to the total input of iron may be in the range of 10000:1 to 1:100, or in the range of 1000:1 to 1:10, or in the range of 10:1 to 1:1. The total input of iron may be in the amount in the range of 0.00001 g/L to 0.5 g/L, or in the range of 0.0001 g/L to 0.1 g/L, or in the range of 0.001 g/L to 0.05 g/L.

The media may contain at least one shear protectant of: polyethylene glycol (PEG), Pluronic F68, Pluronic F127, methyl cellulose (MC), (hydroxypropyl)methyl cellulose (HPMC), hydroxypropyl cellulose (HPC), carboxymethyl cellulose (CMC), dextran sulfate, or any other appropriate shear protectant or their combination. The total input of the shear protectant may be in the range of 0 g/L to 50 g/L, or in the range of 0.02 g/L to 10 g/L, or in the range of 0.1 g/L to 5 g/L.

In one aspect of the invention, the culture medium may comprise a composition of total inputs as described in Table 6.

### Momentary Composition

According to the present invention, the physicochemical parameters and composition of the culture medium may be optimized to facilitate fast biomass production, efficient use of nutrients and low production of waste metabolites.

The osmolality of the medium may be in the range of 200 mOsm/kg to 400 mOsm/kg, or range of 250 mOsm/kg to 350 mOsm/kg, or range of 280 mOsm/kg to 330 mOsm/kg. Osmolality may be adjusted before or after the culture medium is introduced into the cultivation device, or a combination of both, and it may be adjusted at a single time point or multiple timepoints. To increase osmolality, NaCl, KCl, glucose, any other appropriate osmolyte or their combination may be used. To decrease osmolality, water or any other appropriate dilute aqueous solution may be used.

The pH of the culture medium in the cultivation device may be in the range of 6 to 8, or in the range of 6.5 to 7.5, or in the range of 6.8 to 7.3. Adjustment of pH may be performed before or after the culture medium is introduced into the cultivation device, or a combination of both, and it may be adjusted at a single time point or multiple timepoints. NaOH, HCl, NaHCO₃, or any other appropriate acid or base may be used to adjust the pH; alternatively, pH may be adjusted by changing the partial pressure of CO2 in the cultivation device (higher CO2 partial pressure will result in more CO2 being dissolved into the culture medium, leading to lower pH). The partial pressure of CO2 in the cultivation device may be adjusted by changing the percentage of CO2 in the sparging gas, changing the total pressure in the cultivation device, or changing the mixing and sparging rate in the cultivation device (reducing or increasing CO2 mass transfer coefficient), or any other appropriate method. The partial pressure of CO2 in the cultivation device may be in the range of 0.05 kPa to 100 kPa, or in the range 2 kPa to 60 kPa, or in the range 5 kPa to 30 kPa.

The culture medium may comprise a shear protectant to prevent cell damage from mechanical forces caused by mixing and/or sparging in the cultivation device. As a shear protectant may be used at least one of: polyethylene glycol (PEG), methyl cellulose (MC), (hydroxypropyl)methyl cellulose (HPMC), hydroxypropyl cellulose (HPC), carboxymethyl cellulose (CMC), dextran sulfate, or any other appropriate shear protectant, or their combination. Shear protectants may be present in the culture medium in a concentration in the range of 0 g/L to 50 g/L, or in the range of 0.02 g/L to 10 g/L, or in the range of 0.1 g/L to 5 g/L.

The momentary concentration of sugars in the medium may be in the range 0.005 g/L to 40 g/L, or in the range 0.1 g/L to 20 g/L, or in the range 0.5 g/L to 5 g/L.

The momentary concentration of all amino acids (taking into account both amino acids sourced from the hydrolysate and amino acids added separately and biologically available derivatives, such as esters) and peptides in the medium may be in the range of 0.005 g/L to 30 g/L, or in the range 0.1 g/L to 15 g/L, or in the range 0.5 g/L to 10 g/L.

The composition of culture media as described above may be suitable for cell lines that have been extensively adapted to conditions in vitro. However, some cell types may require additional components in the medium, for example protein growth factors, to survive and proliferate. In another aspect of the invention, a medium composition suitable for these growth-factor dependent cell lines may be described as follows.

The hydrolyzates of plant protein isolates may be used as amino acid sources in culture media according to the invention. Recombinant protein production may be used in culture medium components preparation.

The culture medium according to the invention may comprise macronutrients and micronutrients, other components adjusting the properties of the basal medium (osmolality and availability of micronutrients) and signaling components. The components may be dissolved, for example, in purified water, or in water with inorganic salts, for example phosphate buffer saline (PBS) or water or PBS with Bovine serum albumin (BSA), for example 1% BSA in total.

The signaling compounds may vary according to the specific cell type used in the cultivation in the bioreactor. Examples of those cells may be fibroblasts, myoblasts, adipocytes and their precursors or a combination thereof.

The signaling compounds may or may not induce specific change in the cell fate. Examples of these changes may be stimulation of proliferation and/or stimulation of differentiation. The signaling compounds may be used in a certain order during a certain time period. Examples of those may be the usage of a signaling compound for stimulation of proliferation which is then in the media substituted with the signaling compound for differentiation induction. The precise order of dosing of signaling compounds may or may not be correlated or crosslinked with other tools which affect the cell fate during cultivation.

Signaling compounds for various cell types aimed for stimulation of proliferation may comprise, for example, at least one of the following signaling proteins: FGF family ligands, insulin, insulin like growth factor 1 (IGF-1), TGF family ligands, or transferrin, or any other appropriate signaling compound.

Signaling compounds for various cell types aimed for myogenic differentiation may comprise at least one of FGF, insulin, TGF, Transferrin, IGF, Epidermal growth factor (EGF), Bone morphogenic protein (BMP), Interleukin 6 (IL-6), or IL-13, or any other appropriate signaling compound.

The culture medium according to the invention may comprise amino acids (AA) or their sources, in combination with at least one type of compounds that may be selected from a group comprising: saccharides, fatty acids, vitamins and organic micronutrients, mineral compounds, for example inorganic salts, supplements, for example iron supplementation compounds, organic amines, signaling compounds, for example growth factors or signaling proteins or oligonucleotides, shear protectants, additional compounds, or compounds for manipulation, or any other appropriate compounds, or a combination thereof. The media may also contain other compounds, like phospholipids, or nucleic acids, for example. The amino acids may be sourced, for example, from a protein hydrolysate.

The amino acids and their derivatives that may be supplied to the media are for example: glycine, L-alanine, L-arginine, L-asparagine L-aspartic acid, L-cystine L-glutamic acid, L-glutamine, L-histidine, L-hydroxyproline, L-ornithine, L-citrulline, L-isoleucine, L-leucine, L-lysine, L-methionine, L-phenylalanine, L-proline, L-serine, L-threonine, L-pyroglutamic acid, L-phosphoserine, L-tryptophan, L-tyrosine or L-valine. For the preparation of the culture medium, the given amino acid may be added in the pure form, or as part of a complex mixture of compounds (for example a hydrolysate), or the hydrates or salts (for example hydrochlorides or sodium salts) of amino acids may be used.

The culture media according to present invention may comprise protein hydrolysate as a main source of amino acids. The protein hydrolysate may serve as a source of all important amino acids in culture media according to the invention for the purpose of cell cultivation, or some amino acids may be supplied to the media separately, for example L-methionine, which is found in very low concentrations in most scalable protein sources. Other different individual amino acids may be supplied separately from a different source than a protein hydrolysate.

In one aspect of the invention, the culture medium may comprise at least one of the amino acids listed in Table 4. There is also disclosed in Table 4 the possible exemplary, but not limiting, concentration of at least one amino acid that may be used in the culture medium according to the invention.

TABLE 4

| Amino Acids | Concentration mg/L |
|---|---|
| Glycine | 0-1875 |
| L-Alanine | 0-445 |
| L-Arginine hydrochloride | 0-14750 |
| L-Asparagine-H2O | 0-750 |
| L-Aspartic acid | 0-665 |
| L-Cysteine hydrochloride-H2O | 0-1756 |
| L-Cystine-2HCl | 0-3129 |
| L-Glutamic acid | 0-735 |
| L-Glutamine | 0-36500 |
| L-Histidine hydrochloride-H2O | 0-3148 |
| L-Isoleucine | 0-5447 |
| L-Leucine | 0-5905 |
| L-Lysine hydrochloride | 0-9125 |
| L-Methionine | 0-1724 |
| L-Phenylalanine | 0-3548 |
| L-Proline | 0-1725 |
| L-Serine | 0-2625 |
| L-Threonine | 0-5345 |
| L-Tryptophan | 0-902 |
| L-Tyrosine disodium salt dihydrate | 0-5579 |
| L-Valine | 0-5285 |

The culture medium may comprise at least one of the following organic micronutrient compounds: spermine, spermidine, putrescine, thymidine, L-Ornithine, Ethanolamine, myo-inositol, choline and/or any other appropriate organic micronutrient compounds.

Signaling compounds, for example growth factors, may be used in the culture medium according to the invention. For example, at least one of: transferrin, insulin, FGF (for example FGF-1, FGF-2), TGF (for example TGF beta 1), IGF, or any other appropriate compounds may be used as a signaling compound.

In one aspect of the invention, the content of signaling compounds, for example content of growth factors, such as FGF, TGF beta 1, insulin or transferrin or other signaling compounds may be reduced. The concentration of TGF beta 1 may be in the range of 0 to 0.002 mg/L. The concentration of transferrin in the culture medium according to the invention may be in the range of 0 to 10 mg/L, or in the range of 0.1 to 8 mg/L, or in the range of 0.5 to 5 mg/L. In one aspect of the invention, the reduced amount of transferrin may be in the range of 0 to 0.01 mg/L

The concentration of insulin in the culture medium may be in the range of 0 to 2 g/L, or in the range of 0.1 mg/L to 1 g/L, or 0.5 mg to 500 mg/L. In one aspect of the invention, the reduced amount of insulin may be in the range of 0 to 0.1 mg/L

The concentration of FGF-2 in the culture medium may be in the range of 0 to 1 mg/L, or in the range of 0.1 to 0.8 mg/L, or 0.2 to 0.5 mg/L. In one aspect of the invention, the reduced amount of FGF-2 may be in the range of 0 to 0.01 mg/L.

The concentration of TGF beta 1 in the culture medium may be in the range of 0 to 0.2 mg/L, or in the range of 0.01 to 0.15 mg/L, or 0.05 to 0.1 mg/L. In one aspect of the invention, the reduced amount of TGF beta 1 may be in the range of 0 to 0.001 mg/L.

In one aspect of the invention, the culture medium may be without content of any signaling compounds, for example growth factors. The culture medium according to the invention may be serum free and/or protein free.

The culture medium may comprise anti-foaming agent, for example silicone-based anti-foaming agents, polyethylene glycol (PEG), poly vinyl alcohol (PVA), polydimethylsiloxane, polysorbate 80, or vegetable oils, or any other appropriate anti-foaming agent, or the combination thereof. The concentration of the anti-foaming agent in the culture medium may be in the range of 0.001% to 5%, or in the range of 0.01 to 1%, or in the range of 0.1 to 0.5% by weight.

In one aspect of the invention, the content of culture medium components may be in the ranges according to Table 5.

TABLE 5
ranges of concentrations of culture medium components

| Media Component | Concentration (mg/L) |
|---|---|
| Supplement | |
| Transferrin | 0-10 |
| Insulin | 0-2000 |
| FGF2 | 0-1 |
| TGF beta 1 | 0-0.2 |
| Sodium selenium | 0-1.4 |
| Ascorbate | 0-6400 |
| Sugars | |
| D-Glucose (dextrose) | 0-315100 |
| Fatty Acids | |
| Linoleic acid | 0-4.2 |
| Lipoic acid | 0-10.5 |
| Amino Acids | |
| Glycine | 0-1875 |
| L-Alanine | 0-445 |
| L-Arginine hydrochloride | 0-14750 |
| L-Asparagine-H2O | 0-750 |
| L-Aspartic acid | 0-665 |
| L-Cysteine hydrochloride-H2O | 0-1756 |
| L-Cystine-2HCl | 0-3129 |
| L-Glutamic acid | 0-735 |
| L-Glutamine | 0-36500 |
| L-Histidine hydrochloride-H2O | 0-3148 |
| L-Isoleucine | 0-5447 |
| L-Leucine | 0-5905 |
| L-Lysine hydrochloride | 0-9125 |
| L-Methionine | 0-1724 |
| L-Phenylalanine | 0-3548 |
| L-Proline | 0-1725 |
| L-Serine | 0-2625 |
| L-Threonine | 0-5345 |
| L-Tryptophan | 0-902 |
| L-Tyrosine disodium salt dihydrate | 0-5579 |
| L-Valine | 0-5285 |
| Vitamins | |
| Biotin | 0-0.35 |
| Choline chloride | 0-898 |
| D-Calcium pantothenate | 0-224 |
| Folic acid | 0-265 |
| i-Inositol | 0-1260 |
| Niacinamide | 0-202 |
| Pyridoxine hydrochloride | 0-201.3 |
| Riboflavin | 0-21.9 |
| Thiamine hydrochloride | 0-217 |
| Vitamin B12 | 0-68 |
| Inorganic Salts | |
| Calcium chloride (CaCl2) (anhyd.) | 0-11660 |
| Cupric sulphate (CuSO4—5H2O) | 0-0.13 |
| Ferric nitrate (Fe(NO3)3—9H2O) | 0-5 |
| Ferric sulphate (FeSO4—7H2O) | 0-41.7 |
| Magnesium chloride (anhyd.) | 0-2864 |
| Magnesium sulphate (MgSO4) (anhyd.) | 0-4884 |
| Potassium chloride (KCl) | 0-31180 |
| Sodium bicarbonate (NaHCO3) | 0-243800 |
| Sodium chloride (NaCl) | 0-699550 |
| Sodium phosphate dibasic (Na2HPO4) (anhyd.) | 0-7102 |
| Sodium phosphate monobasic (NaH2PO4—H2O) | 0-6250 |
| Zinc sulphate (ZnSO4—7H2O) | 0-43.2 |
| Additional compounds | |
| Hypoxanthine Na | 0-239 |
| Putrescine 2HCl | 0-8.1 |
| Sodium pyruvate | 0-5500 |
| Thymidine | 0-36.5 |

In other aspects of the invention, the culture medium may comprise signaling molecules or nucleic acids.

### Nucleic Acids—Oligonucleotides

In one aspect of the invention, oligonucleotides may be used as constituent components of a culture medium for a cultivation of cells. Oligonucleotides may be with single or double stranded chains of nucleic acids containing 10 to 70 nucleotides or 10 to 120 or 1 to 1,000 nucleotides.

In one aspect of the invention, the oligonucleotides may be added to the culture medium in molar concentration in the range of 5 to 100 nM/L, or in the range of 5 to 500 nM/L, or in the range of 50 nM/L to 50 mM/L, or the concentration may vary during the cultivation, when a peak of higher concentration may be followed with the lower concentration. The peak of high concentration may be from 1-10 hours or 10-72 hours of the cultivation.

In one aspect of the invention oligonucleotides may be a one of the components of a cell type specific signaling compound or may be added to the culture medium independently to the other components.

Examples of oligonucleotides serving as AONs may be oligonucleotides whose target are mRNA of target genes. Examples of those target genes may be ferroportin, myostatin, p53, miRNA140 or others.

Examples of oligonucleotides serving as ligand to the suitable protein (aptamers) may be oligonucleotides able to bind the target proteins such as FGF-2 receptor, TGF-beta receptor, TrF receptor, insulin receptor or others.

As additional compound may be used at least one of: hypoxanthine, putrescine, pyruvate, thymidine, ethanolamine the salts or derivatives thereof, for example sodium hypoxanthine or putrescine dihydrochloride, or any other appropriate additional compound.

The hypoxanthine, for example hypoxanthine sodium, may be used in the culture medium according to the invention in the concentration in the range of 0 to 239 mg/L, or in the range of 10 to 200 mg/L, or in the range of 50 to 100 mg/L.

The putrescine, for example putrescine dihydrochloride, may be used in the culture medium according to the invention in the concentration in the range of 0 to 8.1 mg/L, or in the range of 1 to 6 mg/L, or in the range of 2 to 5 mg/L.

The pyruvate, for example pyruvate sodium, may be used in the culture medium according to the invention in the concentration in the range of 0 mg/L to 5.5 g/L, or in the range of 100 mg/L to 3 g/L, or in the range of 500 mg/L to 1 g/L.

The thymidine may be used in the culture medium according to the invention in the concentration in the range of 0 to 36.5 mg/L, or in the range of 5 to 25 mg/L, or in the range of 10 to 20 mg/L.

The recombinantly prepared signaling compounds may be used in the culture medium according to the invention. The signaling compounds may be stabilized to prevent degradation, for example thermal degradation or proteolytic degradation. They may be secreted into the culture medium, or accumulated in the cellular or subcellular compartment. Then in the process of harvesting they may be or may not be collected, purified and separated or whole culture may be collected. From the whole cultivated culture, various fractions (parts) may be divided and collected in a form of pellets that are easy to handle. Those pellets may be further processed and may serve as a direct compound to be added to the culture medium. Pellets may be dissolved, lysed or reconstituted prior the application into the culture medium in an appropriate solvent.

In one aspect of the invention, a production of recombinant signaling compounds may be used as culture medium components. The recombinant protein production may comprise the following expression systems: bacterial (for example Escherichia coli, Bacillus subtilis), Brewer's yeast (Saccharomyces cerevisiae), non-conventional yeast (for example Pichia pastoris, Hansenula polymorpha, Yarrowia lipolytica), filamentous fungi (for example Aspergillus spp., Trichoderma reesei), plants (for example Nicotiana tabacum, Hordeum vulgare, Zea May), insect cells or mammalian cell lines (for example HEK293, CHO-K1), or any other appropriate expression systems. The recombinant protein production followed by the cellular lysis and derivation of the pellets or other recombinant protein rich derivatives may be used for example in Streptococcus thermophilus, S. cerevisiae, P. pastoris and various strains of species Lactobacillus spp. such as Lactobacillus acidophilus, Lactobacillus plantarum, and Lactobacillus casei.

In one aspect of the invention, the culture medium for cell cultivation for preparing food products may have the total input of hydrolysate expressed as dry protein weight introduced into the culture medium within the cultivation process in the range of 8 g/L to 50 g/L.

The total input of amino acids from hydrolysate, including amino acids in the form of short peptides or suitable bioavailable derivatives may be at least 75% by weight of the total input of all amino acids into the culture medium.

The source of substrate for hydrolysis may be selected from at least one of: phototrophic organisms, such as land plants, green algae, red algae, brown algae, or other phototrophic eukaryotes, phototrophic prokaryotes such as cyanobacteria, or cultivated heterotrophic prokaryotes or eukaryotes, such as bacteria or yeast.

The source of substrate for hydrolysis may be selected from at least one of: soy, pea, rice, wheat, corn, fava beans, alfalfa, hemp, chickpea, potato, pumpkin, rapeseed, red lentil, Spirulina, Chlorella, sunflower, water lentil, mung bean or baker's yeast.

The source of substrate for hydrolysis may be at least one of: soy bean, fava bean, or pea.

In one aspect of the invention, the culture medium may further comprise supplemented at least one of: L-methionine, or L-cysteine, L-cystine, L-ornithine, L-tryptophan, L-histidine, L-threonine, added separately from the hydrolysate.

The total input of amino acids added to the culture medium separately from the hydrolysate may be in the range of 0.1 g/L to 10 g/L.

The total input of amino acids added to the culture medium separately from the hydrolysate may be in the range of 0.5 to 15% expressed as a percentage of the total input of hydrolysate protein into the culture medium.

The total amount of L-cysteine in culture medium may be in the range of 1 to 5% by weight with respect to the total amount of hydrolysate protein in the culture medium.

The total amount of L-ornithine in culture medium may be in the range of 0.001 to 0.5% with respect to the total amount of hydrolysate protein in the culture medium.

The total amount of L-methionine in culture medium may be in the range of 0.2 to 2% with respect to the total amount of hydrolysate protein in the culture medium.

The total amount of L-tryptophan in culture medium may be in the range of 0.2 to 2% with respect to the total amount of hydrolysate protein in the culture medium.

The total amount of L-histidine in culture medium may be in the range of 0.15 to 1.5% with respect to the total amount of hydrolysate protein in the culture medium.

The total amount of L-threonine in culture medium may be in the range of 0.3 to 3% with respect to the total amount of hydrolysate protein in the culture medium.

In one aspect of the invention, the culture medium may further comprise at least one compound from a group consisting of: vitamins, sugars, minerals, organic amines, micronutrients, iron supplementation compounds, shear protectants and low-abundance organic compounds, or a combination thereof.

The vitamin may comprise at one least of: alpha-tocopherol (vitamin E), ascorbic acid (vitamin C), vitamin B12, biotin, choline, pantothenic acid, folic acid, niacinamide, pyridoxine, riboflavin, thiamine, i-inositol, or their derivatives, or a combination thereof.

The sugar may comprise at least one of: glucose, fructose, galactose, sucrose, lactose, maltose, or a combination thereof.

The organic amine may comprise at least one of: putrescine or ethanolamine.

The micronutrient may comprise at least one of: spermine, spermidine, putrescine, thymidine, L-Ornithine, Ethanolamine, myo-inositol, or choline.

The iron supplementation compound may comprise at least one compound in oxidation state iron(III) or iron(II).

The shear protectant may comprise at least one of: polyethylene glycol (PEG), Pluronic F68, Pluronic F127, methyl cellulose (MC), (hydroxypropyl)methyl cellulose (HPMC), hydroxypropyl cellulose (HPC), carboxymethyl cellulose (CMC), or dextran sulfate, or a combination thereof.

In one aspect of the invention, the total input of vitamins per liter of culture medium, except the vitamins present in lysates or extracts, may be in the range of 20 mg/L to 300 mg/L, the total input of sugars per liter of culture medium may be in the range of 3 g/L to 20 g/L, the total input of iron per liter of culture medium may be in the range of 0.001 g/L to 0.05 g/L.

The total input of choline may be in the range of 20 mg/L to 500 mg/L.

The total input of niacinamide, or another vitamer of vitamin B3, may be in the range of 6 mg/L to 100 mg/L.

The total input of mineral compounds introduced into the cultivation process may be in the range of 1 g/L to 20 g/L.

The total input of Na⁺ may be in the range of 30 to 100 mmol/L.

The total input of Cl⁻ may be in the range of 35 to 110 mmol/L.

The total input of Mg²⁺ is in the range of 0.5 to 8 mmol/L.

The total input of PO₄³⁻ is in the range of 0.7 to 10 mmol/L.

The total input of SO₄²⁻ is in the range of 0.3 to 3 mmol/L.

The total input of K⁺ is in the range of 4 to 15 mmol/L.

The total input of the shear protectant may be in the range of 0.1 g/L to 5 g/L.

In one aspect of the invention, the culture medium may comprise exogenous signaling protein in low concentration. The concentration of exogenous signaling protein may be 0, or the concentration of exogenous signaling protein may be in the range of 0 to 50 mg/L.

The cells for cultivation may be non-human metazoan cells.

In one aspect of the invention, the cells for cultivation may be non-human vertebrate cells.

In one aspect of the invention the cells for cultivation may be selected from:
stem cells comprising embryonic stem cells (ESCs) and other cell types derived from blastocysts or other early-stage embryos;
muscle stem cells, such as myosatellite cells, mesenchymal stem cells, or cells where stem characteristics are established in vitro, such as induced pluripotent stem cells (iPSCs);
or cells with the characteristics of: myoblasts, myocytes, fibroblasts, myofibroblasts, fibro-adipogenic progenitors, preadipocytes, adipocytes, epithelial cells, chondroblasts, chondrocytes, macrophages, keratinocytes, hepatocytes, or Sertoli cells.

In one aspect of the invention the cells for cultivation may be anchorage independent, meaning that the cells are able to survive and grow in suspension conditions without attachment to any surface, and/or the cells for cultivation may be able to survive and grow as a suspension of cell clumps, cell aggregates, spheroids or organoids, or the combination thereof.

The culture medium according to the invention may be used for suspension cultivation, meaning that the cultivation process entails cultivating the cells in a stirred tank or other suitable bioreactor type, where the majority of cells is present as a suspension of single cells or cell aggregates.

In one aspect of the invention the cells for cultivation may be immortalized.

In one aspect of the invention the pH of the culture medium may be in the range of 6.8 to 7.3.

In one aspect of the invention the culture medium may comprise a source of bioavailable inorganic nitrogen, wherein the total input of inorganic nitrogen source may be in the range of 1 g/L to 10 g/L.

In one aspect of the invention the process for preparation of the culture medium may have the characteristics of continuous medium preparation, where the medium components may be introduced into the cultivation process separately, the process may comprise the following steps:
a) introduction of a concentrated basal medium into the cultivation device at a defined flow rate, wherein the basal medium is X times more concentrated than the desired concentration in the final medium and the basal medium is introduced at a flow rate 1/X times the total output flow rate from the cultivation device
b) introduction of a concentrated sugar solution into the cultivation device, wherein the flow rate is controlled so that the concentration of the sugar in the cultivation medium is kept at a desirable setpoint,
c) introduction of a solution of hydrolysate and amino acids into the cultivation device, wherein the flow rate is controlled so that the concentration of amino acids in the medium is kept at a desirable setpoint,
d) introduction of a concentrated solution of NaCl into the cultivation device, wherein the flow rate is controlled so that the total osmolality of the medium is kept at a desirable setpoint, for example 310 mOsm/kg,
e) introduction of a demineralized water into the cultivation device, wherein the flow rate is adjusted to be equal to the difference of the total output flow rate from the cultivation device and the sum of all other input flow rates, thereby keeping a constant volume of liquid in the bioreactor.

In one aspect of the invention the process for preparation of the culture medium may comprise a pH adjustment by changing the partial pressure of CO2.

In one aspect of the invention the process for preparation of the culture medium comprising protein hydrolysate may comprise the step of hydrolysis of the source of protein substrate selected from at least one of: phototrophic organisms, such as land plants, green algae, red algae, brown algae, or other phototrophic eukaryotes, phototrophic prokaryotes such as cyanobacteria, or cultivated heterotrophic prokaryotes or eukaryotes, such as bacteria or yeast, or the source of protein substrate for hydrolysis may be selected from at least one of: soy, pea, rice, wheat, corn, fava beans, alfalfa, hemp, chickpea, potato, pumpkin, rapeseed, red lentil, Spirulina, Chlorella, sunflower, water lentil, mung bean or yeast.

The concentration of protein in the reaction mixture may be 30 to 130 g/L.

In one aspect of the invention the protein substrate for hydrolysis may be subjected to an initial thermal pretreatment to improve solubility and susceptibility to hydrolysis, wherein the temperature may be in the range of 80 to 95° C. for 15 to 60 minutes.

In one aspect of the invention the process for preparation of the culture medium may comprise preparation of the protein hydrolysate by enzymatic hydrolysis, wherein the enzymes used for the hydrolysis may comprise at least one endoprotease and at least one exoprotease.

In one aspect of the invention the process for preparation of the culture medium may comprise hydrolysis using at least one of the following types of enzymes: serine protease (for example a subtilisin-like protease), cysteine protease, metalloprotease, glutamic protease or aspartic protease.

The process of preparation of the culture medium may comprise hydrolysis using at least one enzyme of: Alcalase, Flavourzyme, Protamex, Novo-Pro D, Thermoase PC10FNA, Protease AN Amano 100SD, Protease A Amano 2SD, Protease M Amano SD, Protease P Amano 6SD, ProteAX, Peptidase R, Alkaline Protease, Corolase 7089, Corolase 2TSN, Corolase 8000, Maxipro TNP, Maxipro FPC, Papain, or Bromelain.

The concentration of the enzyme may be in the range of 0.05 to 5% expressed as a ratio of the concentration of enzyme to the concentration of substrate protein in the reaction mixture.

The process of enzymatic hydrolysis may take place at a temperature in the range of 30 to 65° C. The pH may be in the range of 5 to 9 and time range may be 10 to 26 hours.

The enzyme may be immobilized on microcarriers.

In one aspect of the invention in the process for preparation of the culture medium the amount of the source protein converted into free amino acids, expressed as mass concentration of amino acids to mass concentration of protein may be in the range of 35 to 65%.

The degree of hydrolysis defined as the percentage of hydrolyzed peptide bonds out of the total amount of peptide bonds present in the substrate at the start of the hydrolysis reaction may be in the range of 20% to 50%.

In one aspect of the invention the process for preparation of the culture medium may comprise monitoring of the hydrolysis by conductometry.

In one aspect of the invention in the process for preparation of the culture medium the hydrolysate may be further processed with phytase to remove phytic acid.

In one aspect of the invention the hydrolysate may be thermally treated at the end of hydrolysis in order to deactivate enzymes and kill microorganisms.

The temperature of the thermal treatment of the hydrolysate may be in the range of 85 to 100° C. for time in the range of 20 to 120 min, or the temperature may be in the range of 100 to 155° C. for time in the range of 3 to 300 s.

In the process of cell cultivation according to the invention, the culture medium according to the description above may be used.

In one aspect of the invention, the cells used in the process of cell cultivation may be non-human metazoan cells.

In one aspect of the invention, the cells used in the process of cell cultivation may exhibit at least one of the following properties:
a) immortalization (absence of Hayflick limit),
b) ability to survive and proliferate in the absence of attachment to a solid surface (anchorage independence),
c) ability to synthesize glutamine,
d) ability to synthesize asparagine,
e) ability to synthesize proline,
f) ability to survive and proliferate in the absence of growth factors, or
g) ability to uptake iron in the absence of transferrin.

The culture medium according to the above mentioned description may be used for cell cultivation for the purpose of preparing the food product.

EXAMPLE'S

### Example 1—Enzymatic Hydrolysis by Free Enzyme

Hydrolysis by free enzyme was performed in the hydrolysis tank by dissolving soy protein isolate in distilled water to a concentration of 10 g/L and the addition of Alcalase to a concentration of 0.05 g/L. The Alcalase used was supplied by Novozymes company. The resulting solution had a basic pH, allowing for a high activity of Alcalase at 62° C. Over 2 hours with constant mixing, the pH of the solution decreased as the results of the hydrolysis of peptide bonds and increased number of carboxylic groups. These conditions allowed for a high activity of Flavourzyme, which was added to a concentration of 0.15 g/L. The resulting mixture was then incubated for an additional 20 hours at 62° C. with constant mixing, after which the residual enzyme was thermally deactivated. With this procedure, 43% of the source protein was converted into free amino acids.

Results of HPLC analysis of amino acid content using UV detection (cysteine was not measured in this analysis) are summarized in Table 6.

TABLE 6
HPLC analysis of amino acid content in hydrolysate

| Amino acid | mg/L |
|---|---|
| Asp | 136.45 |
| Glu | 253.40 |
| Asn | 388.74 |
| Ser | 248.59 |
| Gln | 170.42 |
| His | 147.57 |
| Gly | 80.27 |
| Thr | 226.47 |
| Arg | 426.90 |
| Ala | 138.12 |
| Tyr | 210.82 |
| Met | 56.30 |
| Val | 286.39 |
| Cystine | 8.46 |
| Trp | 60.30 |
| Phe | 296.79 |
| Ile | 260.56 |
| Leu | 469.37 |
| Lys | 381.21 |
| Pro | 31.25 |
| Sum | 4288.91 |

### Example, 2—Enzymatic Hydrolysis by Immobilized Enzyme

The immobilized enzymes were prepared by suspending 600 mg of NH₂-functionalized porous silica microbeads in 50 ml of distilled water. Silica beads were further activated with the addition of 0.003% by volume of glutaraldehyde. After 30 minutes, excess glutaraldehyde was washed away with distilled water and the silica beads were suspended in half the original volume. The Alcalase, supplied by Novozymes company, was then added to a final concentration of 0.1% with constant stirring. This procedure immobilized 80% of the used enzyme on the silica beads, corresponding to 4 grams of enzyme immobilized per 1 kilogram of silica beads.

The silica beads with immobilized Alcalase were added to a mixture of 13 g/L of a soy protein in distilled water at a density of 10 grams of beads per liter. After hydrolysis in the hydrolysis tank for 2 hours at 62° C. with constant mixing, the beads with Alcalase were removed by centrifugation and 40 grams of silica beads with immobilized Flavourzyme were added. After further hydrolysis for 20 hours at 62° C. with constant mixing, the Flavourzyme beads were removed by centrifugation and the resulting hydrolysate was thermally sterilized for 20 minutes at 130° C. and pressure of 2.5 atmospheres, which also deactivated any enzyme that may have detached from the solid support. After filtration to remove solid debris, the hydrolysate was used to prepare culture media. With this method, 5% of the source protein was converted into free amino acids.

Results of HPLC (UV detection) analysis of amino acid content (cysteine was not measured in this analysis) are summarized in Table 7.

TABLE 7
HPLC analysis of amino acid content in hydrolysate

| Amino acid | Soy, water (mg/L) |
|---|---|
| Asp | 15.33 |
| Glu | 0.00 |
| Asn | 1.51 |
| Ser | 5.86 |
| Gln | 10.57 |
| His | 14.04 |
| Gly | 68.39 |
| Thr | 0.00 |
| Arg | 21.31 |
| Ala | 32.71 |
| Tyr | 36.01 |
| Met | 11.58 |
| Val | 36.01 |
| Cystine | 4.33 |
| Trp | 8.08 |
| Phe | 123.73 |
| Ile | 33.87 |
| Leu | 204.85 |
| Lys | 51.05 |
| Pro | 1.35 |
| Sum | 680.59 |

### Example 3—Culture Media Composition

The culture media have been prepared with the compositions according to the table 8 and table 9.

TABLE 8
example total input of relevant compounds into
the culture medium according to the invention.

| | | concen- |
|---|---|---|
| | | tration |
| | | in media |
| Compound | category | [mg/L] |
| Biotin | vitamins and low- | 0.0035 |
| | abundance organic | |
| | compounds | |
| Choline chloride | vitamins and low- | 8.9800 |
| | abundance organic | |
| | compounds | |
| D-Calcium pantothenate | vitamins and low- | 2.2400 |
| | abundance organic | |
| | compounds | |
| Folic Acid | vitamins and low- | 2.6500 |
| | abundance organic | |
| | compounds | |
| Niacinamide | vitamins and low- | 2.0200 |
| | abundance organic | |
| | compounds | |
| Pyridoxine hydrochloride | vitamins and low- | 2.0130 |
| | abundance organic | |
| | compounds | |
| Riboflavin | vitamins and low- | 0.2190 |
| | abundance organic | |
| | compounds | |
| Thiamine hydrochloride | vitamins and low- | 2.1700 |
| | abundance organic | |
| | compounds | |
| Vitamin B12 | vitamins and low- | 0.6800 |
| | abundance organic | |
| | compounds | |
| i-Inositol | vitamins and low- | 12.6000 |
| | abundance organic | |
| | compounds | |
| Calcium Chloride (CaCl₂) | mineral compounds | 116.6000 |
| (anhydrous) | | |
| Cupric sulfate (CuSO₄•5H₂O) | mineral compounds | 0.0800 |
| Ferric Nitrate (Fe(NO₃)₃•9H₂O) | mineral compounds | 0.0500 |
| Ferrous sulfate (FeSO₄•7H₂O) | mineral compounds | 0.4170 |
| Magnesium Chloride (anhydrous) | mineral compounds | 28.6400 |
| Magnesium Sulfate (MgSO₄) | mineral compounds | 48.8400 |
| (anhydrous) | | |
| Potassium Chloride (KCl) | mineral compounds | 311.8000 |
| Sodium Bicarbonate (NaHCO₃) | mineral compounds | 2,438.0000 |
| Sodium Chloride (NaCl) | mineral compounds | 6,995.5000 |
| Sodium Phosphate dibasic | mineral compounds | 71.0200 |
| (Na₂HPO₄) (anhydrous) | | |
| Sodium Phosphate monobasic | mineral compounds | 62.5000 |
| (NaH₂PO₄•H₂O) | | |
| Zinc sulfate (ZnSO₄•7H₂O) | mineral compounds | 0.4320 |
| Sodium selenite | mineral compounds | 0.0300 |
| Soy protein enzymatic hydrolysate | amino acids | 1,000.0000 |
| (expressed as protein dry mass) | | |
| Ferric citrate | iron | 120.0000 |
| | supplementation | |
| Glucose | sugars | 3150.0000 |
| Putrescine | organic amines | 1.0000 |
| Ethanolamine | organic amines | 3.0000 |

TABLE 9
example momentary composition of relevant compounds
in the culture medium according to the invention

| | | concen- |
|---|---|---|
| | | tration |
| | | in media |
| Compound | category | [mg/L] |
| Biotin | vitamins and low- | 0.0035 |
| | abundance organic | |
| | compounds | |
| Choline chloride | vitamins and low- | 8.9800 |
| | abundance organic | |
| | compounds | |
| D-Calcium pantothenate | vitamins and low- | 2.2400 |
| | abundance organic | |
| | compounds | |
| Folic Acid | vitamins and low- | 2.6500 |
| | abundance organic | |
| | compounds | |
| Niacinamide | vitamins and low- | 2.0200 |
| | abundance organic | |
| | compounds | |
| Pyridoxine hydrochloride | vitamins and low- | 2.0130 |
| | abundance organic | |
| | compounds | |
| Riboflavin | vitamins and low- | 0.2190 |
| | abundance organic | |
| | compounds | |
| Thiamine hydrochloride | vitamins and low- | 2.1700 |
| | abundance organic | |
| | compounds | |
| Vitamin B12 | vitamins and low- | 0.6800 |
| | abundance organic | |
| | compounds | |
| i-Inositol | vitamins and low- | 12.6000 |
| | abundance organic | |
| | compounds | |
| Calcium Chloride (CaCl₂) | mineral compounds | 116.6000 |
| (anhydrous) | | |
| Cupric sulfate (CuSO₄•5H₂O) | mineral compounds | 0.0800 |
| Ferric Nitrate (Fe(NO₃)₃•9H₂O) | mineral compounds | 0.0500 |
| Ferrous sulfate (FeSO₄•7H₂O) | mineral compounds | 0.4170 |
| Magnesium Chloride (anhydrous) | mineral compounds | 28.6400 |
| Magnesium Sulfate (MgSO₄) | mineral compounds | 48.8400 |
| (anhydrous) | | |
| Potassium Chloride (KCl) | mineral compounds | 311.8000 |
| Sodium Bicarbonate (NaHCO₃) | mineral compounds | 2,438.0000 |
| Sodium Chloride (NaCl) | mineral compounds | 6,995.5000 |
| Sodium Phosphate dibasic | mineral compounds | 71.0200 |
| (Na₂HPO₄) (anhydrous) | | |
| Sodium Phosphate monobasic | mineral compounds | 62.5000 |
| (NaH₂PO₄•H₂O) | | |
| Zinc sulfate (ZnSO₄•7H₂O) | mineral compounds | 0.4320 |
| Sodium selenite | mineral compounds | 0.0300 |
| Soy protein enzymatic hydrolysate | amino acids | 1,000.0000 |
| (expressed as protein dry mass) | | |
| Ferric citrate | iron | 120.0000 |
| | supplementation | |
| Glucose | sugars | 3150.0000 |
| Putrescine | organic amines | 1.0000 |
| Ethanolamine | organic amines | 3.0000 |

### Example 4—Culture Medium Batch Mixing

Medium components for batch mixing were prepared. Solution 1 comprising vitamins and micronutrient components was prepared by dissolving the compounds according to table 10 in distilled water, so that the final volume of the solution was 10 L.

TABLE 10

| Compound | mg |
|---|---|
| Biotin | 3.5 |
| Choline chloride | 8980 |
| D-Calcium pantothenate | 2240 |
| Folic Acid | 2650 |
| Niacinamide | 2020 |
| Pyridoxine hydrochloride | 2013 |
| Riboflavin | 219 |
| Thiamine hydrochloride | 2170 |
| Vitamin B12 | 680 |
| i-Inositol | 12600 |
| Cupric sulfate (CuSO₄•5H₂O) | 80 |
| Ferric Nitrate (Fe(NO₃)₃•9H₂O) | 50 |
| Sodium selenite | 30 |
| Ferric citrate | 120000 |
| Putrescine | 1200 |
| Ethanolamine | 3200 |

Solution 2 (Basal solution) was prepared by dissolving compounds according to table 11 in distilled water, so that the final volume of the solution was 80 L.

TABLE 11

| Compound | g |
|---|---|
| Calcium Chloride (CaCl2) (anhydrous) | 116.6 |
| Ferrous sulfate (FeSO4,7H2O) | 0.417 |
| Magnesium Chloride (anhydrous) | 28.64 |
| Magnesium Sulfate (MgSO4) (anhydrous) | |
| | 48.84 |
| Potassium Chloride(KCl) | 311.8 |
| Sodium Bicarbonate (NaHCO3) | 2438 |
| Sodium Chloride (NaCl) | 6995 |
| Sodium Phosphate dibasic (Na2HPO4) (anhydrous) | 71.02 |
| Sodium Phosphate monobasic (NaH2PO4,H2O) | 62.5 |
| Zinc sulfate (ZnSO4,7H2O) | 0.432 |
| Glucose | 3150 |

Hydrolysate—10 liters of soy protein hydrolysate with a protein concentration of 10 g/L was prepared according to a suitable hydrolysis procedure, as is described in the chapter on hydrolysate preparation, for example as is described in Example 1.

Media components were mixed: 1 liter of solution 1, 80 liters of solution 2 and 10 liters of hydrolysate were mixed in a 120 L mixing tank.

The pH of the solution was adjusted to 7.2, using 1 M NaOH or 1 M HCl.

Total volume of the solution was adjusted to 100 L using distilled water.

Final media solution was filtered through 0.1 μm candle filters. The sterile medium was stored in a sterile storage tank, which was directly connected to a cultivation device.

### Example 5—Culture Medium Continuous Mixing

Five medium component solutions were prepared (hydrolysate was prepared according to the procedure for preparation of hydrolysate, other solutions were prepared by dissolving the components in distilled water at the required concentration).

Basal medium solution was prepared with the composition according to table 12.

TABLE 12

| Compound | mg/L |
|---|---|
| Biotin | 0.0105 |
| Choline chloride | 26.94 |
| D-Calcium pantothenate | 6.72 |
| Folic Acid | 7.95 |
| Niacinamide | 6.06 |
| Pyridoxine hydrochloride | 6.039 |
| Riboflavin | 0.657 |
| Thiamine hydrochloride | 6.51 |
| Vitamin B12 | 2.04 |
| i-Inositol | 37.8 |
| Calcium Chloride (CaCl2) (anhydrous) | 349.8 |
| Cupric sulfate (CuSO4,5H2O) | 0.24 |
| Ferric Nitrate (Fe(NO3)3,9H2O) | 0.15 |
| Ferrous sulfate (FeSO4,7H2O) | 1.251 |
| Magnesium Chloride (anhydrous) | 85.92 |
| Magnesium Sulfate (MgSO4) (anhydrous) | 146.52 |
| Potassium Chloride (KCl) | 935.4 |
| Zinc sulfate (ZnSO4,7H2O) | 1.296 |
| Sodium selenite | 0.09 |
| Ferric citrate | 360 |
| Putrescine | 3.6 |
| Ethanolamine | 9.6 |

Buffer solution was prepared with the composition according to table 13.

TABLE 13

| Compound | mg/L |
|---|---|
| Sodium Bicarbonate (NaHCO3) | 24380 |
| Sodium Phosphate dibasic (Na2HPO4) (anhydrous) | 710.2 |
| Sodium Phosphate monobasic (NaH2PO4,H2O) | 625 |

Further the soy protein hydrolysate solution was prepared with the concentration of protein of 10 g/L.

Sugar solutions were prepared with the composition according to table 14.

TABLE 14

| Compound | mg/L |
|---|---|
| Glucose | 63000 |

Salt solutions were prepared with the composition according to table 15.

TABLE 15

| Compound | mg/L |
|---|---|
| Sodium Chloride (NaCl) | 139900 |

All component solutions were prepared in individual mixing tanks and equilibrated to a pH of 7.2, using a 1 M solution of NaOH or a 1 M solution of HCl.

All components were filtered into individual sterile storage tanks, using 0.1 μm candle filters.

The sterile medium components were introduced into the cultivation device at volumes of, in order, 0.33, 0.1, 0.1, 0.05 and 0.05 times the working volume of the cultivation device per day (vvd). Additionally, sterile distilled water is introduced into the cultivation device at 0.37 (vvd).

### Example 6: Culture Medium Composition

The culture medium for cultivation of cells was prepared comprising the following types of media components:
a) signaling compounds
b) basal medium compounds
c) nutritional compounds.

The concentrated stock solutions of these three types of media components were prepared and stored individually. Final culture medium was prepared by mixing them together prior to the cultivation of cells in the final concentration per liter according to requested concentration.

One example of the culture media composition is according to Table 16. This culture medium composition comprises nutritional mixture of soy protein hydrolysate, fatty acids and saccharides combined with vitamins, inorganic salts, additional compounds and growth factors.

TABLE 16
medium composition

| Media Component | Concentration (mg/L) |
|---|---|
| Growth factors | |
| Transferrin | 0.100 |
| Insulin | 20.000 |
| FGF2 | 0.100 |
| TGF beta 1 | 0.002 |
| Saccharides | |
| D-Glucose (dextrose) | 3 151.000 |
| Fatty Acids | |
| Linoleic acid | 0.042 |
| Lipoic acid | 0.105 |
| Nutritional mix | |
| soy hydrolysate | 10 000 |
| Vitamins | |
| Biotin | 0.004 |
| Choline chloride | 8.980 |
| D-Calcium pantothenate | 2.240 |
| Folic acid | 2.650 |
| i-Inositol | 12.600 |
| Niacinamide | 2.020 |
| Pyridoxine hydrochloride | 2.013 |
| Riboflavin | 0.219 |
| Thiamine hydrochloride | 2.170 |
| Vitamin B12 | 0.680 |
| Ascorbate | 64.000 |
| Inorganic Salts | |
| Sodium selenium | 0.014 |
| Calcium chloride (CaCl2) (anhyd.) | 116.600 |
| Cupric sulfate (CuSO4—5H2O) | 0.001 |
| Ferric nitrate (Fe(NO3)3—9H2O) | 0.050 |
| Ferric sulfate (FeSO4—7H2O) | 0.417 |
| Magnesium chloride (anhyd.) | 28.640 |
| Magnesium sulfate (MgSO4) (anhyd.) | 48.840 |
| Potassium chloride (KCl) | 311.800 |
| Sodium bicarbonate (NaHCO3) | 2 438.000 |
| Sodium chloride (NaCl) | 6 995.500 |
| Sodium phosphate dibasic (Na2HPO4) (anhyd.) | 71.020 |
| Sodium phosphate monobasic (NaH2PO4—H2O) | 62.500 |
| Zinc sulfate (ZnSO4—7H2O) | 0.432 |
| Additional compounds | |
| Hypoxanthine Na | 2.390 |
| Putrescine 2HCl | 0.081 |
| Sodium pyruvate | 55.000 |
| Thymidine | 0.365 |

TABLE 17
culture medium composition

| Media Component | Concentration (mg/L) |
|---|---|
| Growth factors | |
| Transferrin | 0.100 |
| Insulin | 20.000 |
| FGF2 | 0.100 |
| TGF beta 1 | 0.002 |
| LIF | 0.050 |
| Saccharides | |
| D-Glucose (dextrose) | 1 000.000 |
| Amino Acids | |
| Glycine | 18.750 |
| L-Alanine | 4.450 |
| L-Arginine hydrochloride | 147.500 |
| L-Asparagine-H2O | 7.500 |
| L-Aspartic acid | 6.650 |
| L-Cysteine hydrochloride-H2O | 17.560 |
| L-Cystine-2HCl | 31.290 |
| L-Glutamic acid | 7.350 |
| L-Glutamine | 365.000 |
| L-Histidine hydrochloride-H2O | 31.480 |
| L-Isoleucine | 54.470 |
| L-Leucine | 59.050 |
| L-Lysine hydrochloride | 91.250 |
| L-Methionine | 17.240 |
| L-Phenylalanine | 35.480 |
| L-Proline | 17.250 |
| L-Serine | 26.250 |
| L-Threonine | 53.450 |
| L-Tryptophan | 9.020 |
| L-Tyrosine disodium salt dihydrate | 55.790 |
| L-Valine | 52.850 |
| Fatty Acids | |
| Linoleic acid | 0.042 |
| Lipoic acid | 0.105 |
| Vitamins | |
| Biotin | 0.004 |
| Choline chloride | 8.980 |
| D-Calcium pantothenate | 2.240 |
| Folic acid | 2.650 |
| i-Inositol | 12.600 |
| Niacinamide | 2.020 |
| Pyridoxine hydrochloride | 2.013 |
| Riboflavin | 0.219 |
| Thiamine hydrochloride | 2.170 |
| Vitamin B12 | 0.680 |
| Ascorbate | 64.000 |
| Inorganic Salts | |
| Sodium selenium | 0.014 |
| Calcium chloride (CaCl2) (anhyd.) | 116.600 |
| Cupric sulfate (CuSO4—5H2O) | 0.001 |
| Ferric nitrate (Fe(NO3)3—9H2O) | 0.050 |
| Ferric sulfate (FeSO4—7H2O) | 0.417 |
| Magnesium chloride (anhyd.) | 28.640 |
| Magnesium sulfate (MgSO4) (anhyd.) | 48.840 |
| Potassium chloride (KCl) | 311.800 |
| Sodium bicarbonate (NaHCO3) | 2 438.000 |
| Sodium chloride (NaCl) | 6 995.500 |
| Sodium phosphate dibasic (Na2HPO4) (anhyd.) | 71.020 |
| Sodium phosphate monobasic (NaH2PO4—H2O) | 62.500 |
| Zinc sulfate (ZnSO4—7H2O) | 0.432 |
| Additional compounds | |
| Hypoxanthine Na | 2.390 |
| Putrescine 2HCl | 0.081 |
| Sodium pyruvate | 55.000 |
| Thymidine | 0.365 |

Another example of the culture media composition is according to Table 17. This culture medium composition comprises nutritional mixture of raw food grade amino acids, fatty acids and saccharide D-glucose combined with vitamins, inorganic salts, additional compounds and growth factors.

The culture medium according to the invention may be suitable for cell cultivation, for example in cultivated meat production or pet food production. The advantageous processes of culture media preparation and the process of protein hydrolysis into shorter peptide chains and/or single amino acids are also provided by the present invention.

1. A culture medium for the cultivation of cells for preparing a food product,
wherein the culture medium comprises a protein hydrolysate as a source of amino acids, wherein the protein hydrolysate is prepared by enzymatic hydrolysis of a protein substrate comprising at least one of: soy, pea, rice, wheat, corn, fava beans, alfalfa, hemp, chickpea, potato, pumpkin, rapeseed, red lentil, Spirulina, Chlorella, sunflower, water lentil, mung bean or baker's yeast,
wherein the cells are non-human metazoan cells,
wherein the total input of the protein hydrolysate expressed as dry protein weight introduced into the culture medium for cell cultivation for preparing the food product within the cultivation process is in the range of 8 g/L to 50 g/L.

2. The culture medium according to claim 1,
wherein at least two types of enzymes are used for the enzymatic hydrolysis, wherein the at least two types of enzymes comprise:
i) at least one endoprotease and at least one exoprotease, or
ii) at least one of the following types of enzymes: serine protease, cysteine protease, metalloprotease, glutamic protease or aspartic protease.

3. The culture medium according to claim 1, wherein the total input of amino acids from the protein hydrolysate, including amino acids in the form of peptides or suitable bioavailable derivatives, is at least 75% by weight of the total input of all amino acids in the culture medium.

4. The culture medium according to claim 1, further comprising at least one of: vitamins, sugars, minerals, organic amines, micronutrients, iron supplementation compounds, shear protectants or low-abundance organic compounds.

5. The culture medium according to claim 1, further comprising at least one exogenous signaling protein, wherein the concentration of the exogenous signaling protein is up to 50 mg/L.

6. The culture medium according to claim 5, wherein the culture medium is free of the exogenous signaling protein.

7. The culture medium according to claim 5, wherein the exogenous signaling protein comprises at least one of: an FGF family ligand, insulin, insulin like growth factor 1, TGF family ligand or transferrin.

8. The culture medium according to claim 1, further comprising a source of bioavailable inorganic nitrogen, wherein the total input of the source of bioavailable inorganic nitrogen is in the range of 1 g/L to 10 g/L.

9. A culture medium for the cultivation of cells for preparing a food product,
wherein the culture medium comprises a protein enzymatic hydrolysate as a source of amino acids,
wherein the protein enzymatic hydrolysate is prepared by enzymatic hydrolysis of a source protein substrate for hydrolysis comprising at least one of: soy, pea, rice, wheat, corn, fava beans, alfalfa, hemp, chickpea, potato, pumpkin, rapeseed, red lentil, Spirulina, Chlorella, sunflower, water lentil, mung bean or baker's yeast,
wherein for the enzymatic hydrolysis is used at least one endoprotease enzyme and at least one exoprotease enzyme, wherein the endoprotease is a serine endoprotease,
wherein the cells are non-human metazoan cells,
wherein the total input of the protein enzymatic hydrolysate expressed as dry protein weight introduced into the culture medium for cell cultivation for preparing the food product within the cultivation process is in the range of 8 g/L to 50 g/L.

10. The culture medium according to claim 9,
wherein the culture medium comprises at least one essential amino acid, wherein the conversion efficiency for essential total amino acids is in the range of 30% to 100%, as calculated by equation (1):
$${H}_{EAA} = \frac{\frac{{A}_{EAAM}}{\sum {A}_{EAAM}}}{\frac{{A}_{EAAC}}{\sum {A}_{EAAC}}} * 100 , \qquad ( 1 )$$
where
H_{EAA} is the highest conversion efficiency for a particular amino acid,
A_{EAAM} is the content of that particular essential amino acid in 100 g of protein in the culture medium,
ΣA_{EAAM} is the total content of all essential amino acids in 100 g of protein in the culture medium,
A_{EAAC} is the content of that particular essential amino acid in 100 g of cellular protein, and
I_{AEAAC} is the total content of all essential amino acids in 100 g of cellular protein, wherein the content per 100 g of cellular protein for His is 2.7 g, Ile is 5.1 g, Leu is 8.9 g, Lys is 8.2 g, Met is 2.9 g, Phe is 4.7 g, Thr is 4.8 g, Trp is 1.6 g, Val is 5.8 g, and the sum is 44.7 g.

11. The culture medium according to claim 9, further comprising a supplemented amino acid comprising at least one of: L-methionine, L-cysteine, L-cystine, L-ornithine, L-tryptophan, L-histidine or L-threonine, added separately from the protein enzymatic hydrolysate.

12. The culture medium according to claim 11, wherein the total input of supplemented amino acids added to the culture medium separately from the hydrolysate is in a range of 0.1 g/L to 10 g/L.

13. The culture medium according to claim 9, further comprising at least one of: vitamins, sugars, minerals, organic amines, micronutrients, iron supplementation compounds, shear protectants or low-abundance organic compounds.

14. The culture medium according to claim 13, wherein the culture medium comprises an organic amine, wherein the organic amine is putrescine or ethanolamine.

15. The culture medium according to claim 13, wherein the culture medium comprises a micronutrient, wherein the micronutrient comprises at least one of: spermine, spermidine, putrescine, thymidine, L-ornithine, ethanolamine, myo-inositol or choline.

16. The culture medium according to claim 13, wherein the culture medium comprises at least one iron supplementation compound, wherein the iron supplementation compound comprises iron in oxidation state iron(III) or iron(II).

17. The culture medium according to claim 13, wherein the culture medium comprises a shear protectant, wherein the shear protectant comprises at least one of: polyethylene glycol (PEG), Pluronic F68, Pluronic F127, methyl cellulose (MC), (hydroxypropyl)methyl cellulose (HPMC), hydroxypropyl cellulose (HPC), carboxymethyl cellulose (CMC) or dextran sulfate.

18. A culture medium for the cultivation of cells,
wherein the culture medium comprises a protein enzymatic hydrolysate as a source of amino acids,
wherein the protein enzymatic hydrolysate is prepared by enzymatic hydrolysis of a protein substrate comprising at least one of: soy, pea, rice, wheat, corn, fava beans, alfalfa, hemp, chickpea, potato, pumpkin, rapeseed, red lentil, Spirulina, Chlorella, sunflower, water lentil, mung bean or baker's yeast,
wherein the cells are non-human metazoan cells,
wherein at least two types of enzymes are used for the enzymatic hydrolysis, wherein the at least two types of enzymes comprise:
i) at least one endoprotease and at least one exoprotease, or
ii) at least one of the following types of enzymes: serine protease, cysteine protease, metalloprotease, glutamic protease or aspartic protease,
wherein the total input of protein enzymatic hydrolysate expressed as dry protein weight introduced into the culture medium for cell cultivation for preparing the food product within the cultivation process is in the range of 8 g/L to 50 g/L,
wherein the total input of amino acids from the protein enzymatic hydrolysate, including amino acids in the form of short peptides or suitable bioavailable derivatives, is at least 75% by weight of the total input of all amino acids into the culture medium.

19. The culture medium according to claim 18,
wherein the enzymatic hydrolysis occurs in a hydrolysis reaction mixture comprising the enzymes and the protein substrate,
wherein the total enzyme concentration in the hydrolysis reaction mixture is in the range of 0.05 to 5% expressed as a ratio of the concentration of enzyme to the concentration of the protein substrate in the reaction mixture.

20. The culture medium according to claim 18, wherein the degree of the enzymatic hydrolysis of the protein substrate, defined as the percentage of hydrolyzed peptide bonds out of the total amount of peptide bonds present in the substrate at the start of the hydrolysis reaction, is in the range of 10% to 60%.

21. The culture medium according to claim 18, wherein
the cells are anchorage independent, defined as being able to survive and grow in suspension conditions without attachment to any surface, and/or the cells are able to survive and grow as a suspension of cell clumps, cell aggregates, spheroids, organoids.

22. The culture medium according to claim 18, where the medium is used for suspension cultivation of cells, defined as a cultivation process entailing cultivating the cells in a stirred tank or other suitable bioreactor type, wherein the majority of cells are present as a suspension of single cells or cell aggregates.

23. The culture medium according to claim 18, wherein the pH of the culture medium during the cell cultivation is in the range of 6 to 8.

24. A method for preparing a culture medium for the cultivation of cells for preparing a food product,
wherein the culture medium comprises a protein hydrolysate as a source of amino acids,
the method comprising the step of enzymatic hydrolysis of a protein substrate comprising at least one of: soy, pea, rice, wheat, corn, fava beans, alfalfa, hemp, chickpea, potato, pumpkin, rapeseed, red lentil, Spirulina, Chlorella, sunflower, water lentil, mung bean or baker's yeast,
wherein the cells are non-human metazoan cells,
wherein the total input of the protein hydrolysate expressed as dry protein weight introduced into the culture medium for cell cultivation for preparing the food product within the cultivation process is in the range of 8 g/L to 50 g/L.

25. The method for preparing a culture medium according to claim 24,
wherein at least two types of enzymes are used for the enzymatic hydrolysis, wherein the at least two types of enzymes comprise:
i) at least one endoprotease and at least one exoprotease, or
ii) at least one of the following types of enzymes: serine protease, cysteine protease, metalloprotease, glutamic protease or aspartic protease.

26. The method for preparing the culture medium according to claim 24,
wherein the culture medium is prepared by continuous medium mixing process, the method comprising:
a) introduction of a concentrated basal medium into the cultivation device at a defined flow rate,
wherein the basal medium is X times more concentrated than
the desired concentration in the final medium and the basal medium is introduced at a flow rate 1/X times the total output flow rate from the cultivation device,
b) introduction of a concentrated sugar solution into the cultivation device, wherein the flow rate is controlled such that the concentration of the sugar in the cultivation medium is kept at a desirable setpoint,
c) introduction of a solution of hydrolysate and amino acids into the cultivation device, wherein the flow rate is controlled such that the concentration of amino acids in the medium is kept at a desirable setpoint,
d) introduction of a concentrated solution of NaCl into the cultivation device, wherein the flow rate is controlled such that the total osmolality of the medium is kept at a desirable setpoint,
e) introduction of demineralized water into the cultivation device, wherein the flow rate is adjusted to be equal to the difference of the total output flow rate from the cultivation device and the sum of all other input flow rates, thereby keeping a constant volume of liquid in the bioreactor.

27. The method for preparation of the culture medium according to claim 24,
wherein the enzymatic hydrolysis occurs in a hydrolysis reaction mixture comprising the protein substrate and the enzyme, wherein the protein substrate is at a concentration in the range of 30 to 130 g/L.

28. The method for preparation of the culture medium according to claim 24, wherein the enzymatic hydrolysis occurs in a hydrolysis reaction mixture comprising the protein substrate and the enzyme, wherein the enzyme is at a concentration in the range of 0.05 to 5% expressed as a ratio of the concentration of the enzyme to the concentration of protein substrate in the hydrolysis reaction mixture.

29. The method for preparation of the culture medium according to claim 24, wherein the enzymatic hydrolysis takes place at a temperature in the range of 10 to 90° C., pH in the range of 5 to 9 and for a time in the range of 10 minutes to 24 hours.

30. The method for preparation of the culture medium according to claim 24, wherein
wherein the degree of hydrolysis, defined as the percentage of hydrolyzed peptide bonds out of the total amount of peptide bonds present in the protein substrate at the start of the hydrolysis reaction, is in the range of 20% to 50%.
